(19) United States
(12) Patent Application Publication
(10) Pub. No.: US 2013/0078281 A1
(43) Pub. Date: Mar. 28, 2013

(54) ACTIVATORS OF INNATE IMMUNITY

(71) Applicant: University of Georgia Research Foundation, Inc., Athens, GA (US)

(72) Inventors: Biao HE, Bogart, GA (US); Priya Luthra, Athens, GA (US)

(73) Assignee: University of Georgia Research Foundation, Inc., Athens, GA (US)

(21) Appl. No.: 13/686,155

(22) Filed: Nov. 27, 2012

Related U.S. Application Data
(63) Continuation-in-part of application No. PCT/US2011/038313, filed on May 27, 2011.
(60) Provisional application No. 61/348,891, filed on May 27, 2010.

(51) Int. Cl. C07K 14/115; A61K 39/39

(52) U.S. Cl. CPC C07K 14/115; A61K 39/39
USPC 424/281.1; 536/23.72

(57) ABSTRACT

The present invention includes IFNS activating agents that activate expression of IFN-β, activate NF-κB expression, activate an innate immune response, activate the expression of one or more cytokines, and/or induce the expression of interferon beta (IFN-β) through a RNase L and/or MDA5-dependent pathway. Such IFNS activating agents include single stranded RNAs that encode for conserved region II of the L protein of a negative stranded RNA virus, including, but not limited to, viruses of the family Paramyxoviridae. Also included are methods of making and using such IFNS activating agents and compositions and kits including such IFNS activating agents.

### CONTINUING APPLICATION DATA

This application is a continuation-in-part of International Application No. PCT/US2011/038313, filed May 27, 2011, which claims the benefit of U.S. Provisional Application Ser. No. 61/348,891, filed May 27, 2010, each of which is incorporated by reference herein.

### GOVERNMENT FUNDING

This invention was made with government support under AI070847, K02 065795, and R56 AI081816 awarded by the National Institutes of Health. The Government has certain rights in the invention.

### BACKGROUND

Interferon (IFN) has been approved by the Food and Drug Administration (FDA) for the treatment of several indications, with interferon alpha (IFN-α) approved for the treatment of malignant melanoma, chronic hepatitis C (HCV), hepatitis B (HBV), and some types of leukemia and lymphoma, and interferon beta (IFN-β) approved for the treatment of multiple sclerosis (MS). However, these FDA-approved interferons are biologic products made of proteins which are expensive to produce and have relatively short shelf lives. These interferons are administered systemically, resulting in significant, deleterious side effects inside the human body. Thus, there is a need for improved, cost effective alternatives for the delivery of interferons, including improved methods for the localized delivery of interferons. Further, many viral infections are not easily controlled by existing therapeutic agent and there is a need for broadly effective anti-virals to combat viral infections, including emerging viral infections.

### SUMMARY OF THE INVENTION

The present invention includes a method of activating interferon beta expression in a cell, the method including delivering to the cell an isolated single stranded RNA sequence, the isolated single stranded RNA sequence including a nucleotide sequence that is 90% identical to a nucleotide sequence encoding conserved region II of the L protein of a negative stranded RNA virus, or a fragment thereof.

The present invention includes a method of activating interferon beta expression in a subject, the method including delivering an isolated single stranded RNA sequence, the isolated single stranded RNA sequence including a nucleotide sequence that is 90% identical to a nucleotide sequence encoding conserved region II of the L protein of a negative stranded RNA virus, or a fragment thereof, to the subject.

The present invention includes a method of activating interferon beta expression in a cell, the method including delivering to the cell an isolated single stranded RNA sequence, the isolated single stranded RNA sequence including a nucleotide sequence that is 90% identical to the nucleotide sequence SEQ ID NO:1, or a fragment thereof.

The present invention includes a method of activating interferon beta expression in a subject, the method including delivering an isolated single stranded RNA sequence, the isolated single stranded RNA sequence including a nucleotide sequence that is 90% identical to the nucleotide sequence SEQ ID NO:1, or a fragment thereof, to the subject.

The present invention includes a method of activating NF-κB expression in a cell, the method including delivering to the cell an isolated single stranded RNA sequence, the isolated single stranded RNA sequence including a nucleotide sequence that is 90% identical to a nucleotide sequence encoding conserved region II of the L protein of a negative stranded RNA virus, or a fragment thereof.

The present invention includes a method of activating NF-κB expression in a subject, the method including delivering an isolated single stranded RNA sequence, the isolated single stranded RNA sequence including a nucleotide sequence that is 90% identical to a nucleotide sequence encoding conserved region II of the L protein of a negative stranded RNA virus, or a fragment thereof, to the subject.

The present invention includes a method of activating NF-κB expression in a cell, the method including delivering to the cell an isolated single stranded RNA sequence, the isolated single stranded RNA sequence including a nucleotide sequence that is 90% identical to the nucleotide sequence SEQ ID NO:1, or a fragment thereof.

The present invention includes a method of activating NF-κB in a subject, the method including delivering an isolated single stranded RNA sequence, the isolated single stranded RNA sequence including a nucleotide sequence that is 90% identical to the nucleotide sequence SEQ ID NO:1, or a fragment thereof, to the subject.

The present invention includes a method of treating a viral disease, cancer, and/or an autoimmune disease in a subject, the method including delivering an isolated single stranded RNA sequence to the subject, the isolated single stranded RNA sequence including a nucleotide sequence that is 90% identical to a nucleotide sequence encoding conserved region II of the L protein of a negative stranded RNA virus, or a fragment thereof, wherein the isolated single stranded RNA sequence activates the expression of interferon beta and/or NF-κB in the subject.

The present invention includes a method of treating a viral disease, cancer, and/or an autoimmune disease in a subject, the method including delivering an isolated single stranded RNA sequence to the subject, the isolated single stranded RNA sequence including a nucleotide sequence that is 90% identical to the nucleotide sequence SEQ ID NO:1, or a fragment thereof, wherein the isolated single stranded RNA sequence activates the expression of interferon beta and/or NF-κB in the subject.

The present invention includes a method of activating IFN beta expression through a RNase L and/or MDA5-dependent pathway in a cell, the method including delivering to the cell an isolated single stranded RNA sequence, the isolated single stranded RNA sequence including a nucleotide sequence that is 90% identical to a nucleotide sequence encoding conserved region II of the L protein of a negative stranded RNA virus, or a fragment thereof, to the subject.

The present invention includes a method of activating IFN beta expression through a RNase L and/or MDA5-dependent pathway in a subject, the method including delivering an isolated single stranded RNA sequence, the isolated single stranded RNA sequence including a nucleotide sequence that is 90% identical to the nucleotide sequence SEQ ID NO:1, or a fragment thereof, to the subject.

The present invention includes a composition including as one aspect, one or more antigenic agents, and as a second aspect, an isolated polynucleotide sequence including a nucleotide sequence that transcribes a single stranded RNA sequence including a nucleotide sequence that is 90% identical to the nucleotide sequence SEQ ID NO:1, or a fragment thereof. In some aspects, an antigenic aspect includes a nucleotide sequence encoding an antigen.

The present invention includes a composition including an isolated polynucleotide sequence including a nucleotide sequence that transcribes a single stranded RNA sequence including a nucleotide sequence that is 90% identical to a nucleotide sequence encoding conserved region II of the L protein of a virus of a negative stranded RNA virus, or a fragment thereof. In some aspects, such a composition may be used to activate interferon beta expression and/or activate NF-κB expression in a subject. In some aspects, such a composition may be used to generate an immune response and/or enhance the generation of an immune response. In some aspects, such a compositions maybe used as a vaccine.

The present invention includes a composition including as one aspect, one or more antigenic agents, and as a second aspect, an isolated polynucleotide sequence including a nucleotide sequence that transcribes a single stranded RNA sequence including a nucleotide sequence that is 90% identical to a nucleotide sequence encoding conserved region II of the L protein of a virus of a negative stranded RNA virus, or a fragment thereof. In some aspects, an antigenic aspect includes a nucleotide sequence encoding an antigen. In some aspects, such a composition may be used to activate interferon beta expression and/or activate NF-κB expression in a subject. In some aspects, such a composition may be used to generate an immune response and/or enhance the generation of an immune response. In some aspects, such a compositions maybe used as a vaccine.

The present invention includes an isolated polynucleotide sequence including SEQ ID NO:1, or a fragment thereof, wherein the fragment includes at least 10 consecutive nucleotides of SEQ ID NO:1 and the fragment activates IFN beta expression through a RNase L and/or MDA5-dependent pathway.

The present invention includes a isolated polynucleotide sequence including a nucleotide sequence that is 90% identical to a nucleotide sequence encoding conserved region II of the L protein of a negative stranded RNA virus, or a fragment thereof, wherein the isolated polynucleotide sequence includes a stop codon positioned so that the single stranded RNA is not translated into an amino acid sequence and/or does not include sequences encoding the conserved region I of the L protein of a negative stranded RNA virus.

In some aspects of the methods, compositions, kits, vaccines, and isolated polynucleotide sequences of the present invention, the negative stranded RNA virus is of the family Paramyxoviridae. In some aspects, the virus of the family Paramyxoviridae is selected from human parainfluenza virus 1, human parainfluenza virus 2, human parainfluenza virus 3, human parainfluenza virus 4, parainfluenza virus 5, mumps virus, measles virus, human metapneumovirus, human respiratory syncytial virus, bovine respiratory syncytial virus rinderpest virus, canine distemper virus, phocine distemper virus, Newcastle disease virus, avian pneumovirus, Peste des Petits Ruminants virus (PPRV), Sendai virus, Menangle virus, Tupaia paramyxovirus, Tioman virus, Tuhokovirus 1, Tuhokovirus 2, Tuhokovirus 3, Hendravirus, Nipahvirus, Fer-de-Lance virus, Nariva virus, Salem virus, J virus, Mossman virus, or Beilong virus.

In some aspects of the methods, compositions, kits, vaccines, and isolated polynucleotide sequences of the present invention, the single stranded sequence is a non-naturally occurring sequence. In some aspects, the single stranded sequence includes a stop codon positioned so that the single stranded RNA is not translated into an amino acid sequence

In some aspects of the methods, compositions, kits, vaccines, and isolated polynucleotide sequences of the present invention, the single stranded sequence does not encode conserved region I, III, IV, V, and/or VI of the L protein of a negative stranded RNA virus

In some aspects of the methods, compositions, kits, vaccines, and isolated polynucleotide sequences of the present invention, a fragment includes at least 10 consecutive nucleotides of SEQ ID NO:1 and the fragment activates IFN beta expression through a RNase L and/or MDA5-dependent pathway.

In some aspects of the methods, compositions, kits, vaccines, and isolated polynucleotide sequences of the present invention, delivering a single stranded RNA sequence is by administering a DNA expression vector that transcribes the single stranded RNA sequence.

In some aspects of the methods, compositions, kits, vaccines, and isolated polynucleotide sequences of the present invention, the single stranded RNA sequence is an mRNA with a 5′ cap.

In some aspects of the methods, compositions, vaccines, and isolated polynucleotide sequences of the present invention, delivering a single stranded RNA sequence is by administering a composition including the single stranded RNA sequence.

In some aspects of the methods, compositions, kits, vaccines, and isolated polynucleotide sequences of the present invention, a fragment includes at least 10 consecutive nucleotides of SEQ ID NO:1 and the fragment activates IFN beta expression through a RNase L and/or MDA5-dependent pathway.

In some aspects of the methods, compositions, kits, vaccines, and isolated polynucleotide sequences of the present invention, the polynucleotide sequence includes a stop codon at its 5′ end and does is not translated into an amino acid sequence.

In some aspects of the methods, compositions, kits, vaccines, and isolated polynucleotide sequences of the present invention, delivery is intramuscular, intranasal, intravenous, intreperitoneal, subcutaneous, and/or topical.

In some aspects of the methods, compositions, kits, vaccines, and isolated polynucleotide sequences of the present invention, wherein delivery of a single stranded RNA sequence is regulated by a tissue specific promoter.

In some aspects of the methods, compositions, kits, vaccines, and isolated polynucleotide sequences of the present invention, delivery is to the mucosal membranes of the respiratory tract. In some aspects, delivery is by aerosol.

In some aspects of the methods, compositions, kits, vaccines, and isolated polynucleotide sequences of the present invention, delivery is by aerosol.

In some aspects of the methods, compositions, kits, vaccines, and isolated polynucleotide sequences of the present invention, delivery is to liver, lung, central nervous system, nerves, muscle or tumor. In some aspects, delivery is regulated by a tissue specific promoter, including, but not limited to, a liver specific promoter.

In some aspects of the methods, compositions, vaccines, and isolated polynucleotide sequences of the present invention, delivery of a single stranded RNA sequence is regulated by a liver specific promoter.

In some aspects of the methods, compositions, kits, vaccines, and isolated polynucleotide sequences of the present invention, a subject has been exposed to Hepatitis C.

In some aspects of the methods, compositions, kits, vaccines, and isolated polynucleotide sequences of the present invention, a subject has been exposed to a viral disease.

In some aspects of the methods, compositions, kits, vaccines, and isolated polynucleotide sequences of the present invention, a subject suffers from an autoimmune disease. In some aspects, the autoimmune disease is multiple sclerosis. In some aspects, delivery is intranasal

In some aspects of the methods, compositions, kits, vaccines, and isolated polynucleotide sequences of the present invention, delivery is intranasal administration.

In some aspects of the methods, compositions, kits, vaccines, and isolated polynucleotide sequences of the present invention, the subject suffers from cancer. In some aspects, the cancer is melanoma.

The tetras “comprises” and variations thereof do not have a limiting meaning where these terms appear in the description and claims.

Unless otherwise specified, “a,” “an,” “the,” and “at least one” are used interchangeably and mean one or more than one.

For any method disclosed herein that includes discrete steps, the steps may be conducted in any feasible order. And, as appropriate, any combination of two or more steps may be conducted simultaneously.

### BRIEF DESCRIPTION OF THE FIGURES

FIGS. 1A and 1B. Activation of NF-κB by region II of the L gene in an AKT-independent manner. FIG. 1A demonstrates detection of activation of NF-κB by L region-expressing plasmids using EMSA. Nuclear extracts from cells transfected with empty vector or plasmids encoding L, L-I, or L-II were prepared and incubated with ³²P-labeled NF-κB probe and appropriate competitors, and were resolved on a 6% polyacrylamide gel. Treatments: TNF-α, nuclear extracts from cells treated with 20 ng/ml of TNF-a for three hours; NF-κB DNA primers labeled with ³²P; S (specific competitor), unlabeled NF-κB probe (20-fold excess); NS (non-specific competitor), unlabeled mutant NF-κB probe (20-fold excess). FIG. 1B demonstrates that activation of NF-κB by the L-II region was independent of AKT1. A dual luciferase assay, in which BSR-T7 cells were transfected with a plasmid encoding a firefly luciferase gene (F-Luc) under the control of NF-κB-responsive elements, and a plasmid encoding PIV5 L, L-II, L-II mut, or L-I-II proteins, along with a plasmid encoding a Renilla luciferase (R-Luc) as an indicator of transfection efficiency, was performed in the presence of an AKT IV inhibitor (Inh) (0.5 M) (Calbiochem) or vehicle [dimethyl sulfoxide (DMSO)] (Left Panel). Ratios of F-Luc to R-Luc are used as an indicator of reporter gene activity. These ratios were normalized to the activity of the vector alone. All transfections were carried out in replicates of four and error bars represent standard deviation (SD). All P values were calculated using paired t test and are shown in the figure Inhibition of L-activated NF-κB activity by DN AKT1 is shown in right panel.

FIGS. 2A and 2B. The L-II RNA activated NF-κB. FIG. 2A demonstrates the activation of NF-κB by the L-II mut. The L-II mutant contains a stop codon in place of the start codon of L-II. A reporter gene assay was performed as described in FIG. 1. FIG. 2B demonstrates activation of NF-κB by L-II RNA is independent of AKT1. A dual luciferase experiment was performed using AKT1 inhibitor (Inh) (left panel) or AKT1 DN (right panel) along with L, L-II, L-II mut, or L-I-II plasmids as described in FIG. 1. All transfections were carried out in replicates of four and error bars indicate SD.

FIGS. 3A to 3H. The L-II RNA activated IFN-β expression. FIG. 3A demonstrates activation of IFN-β promoter by the L-II mut. A dual luciferase assay was performed as described in FIG. 1. A plasmid containing F-Luc under control of an IFN-β promoter was used in place of the NF-κB-containing promoter described in FIG. 1. FIG. 3B demonstrates induction of IFN-β production by L-II RNA. Plasmids encoding L-II or L-II mut were transfected into 293T cells and the amount of IFN-β in the media were measured using ELISA at one day post-transfection. For all graphs showing concentrations of IFN-β using ELISA, the graph is the average of three independent experiments and error bars represent SD. FIG. 3C demonstrates IFN-β production induced by purified RNA. Vero cells were transfected with empty vector or plasmids containing L-II mut, or infected with wild-type (WT) PIV5, rPIV5VΔC, mock-infected, or transfected with poly(I):poly©. Total RNAs were purified from transfected or infected cells. The purified RNAs were then transfected into 293T cells and concentrations of IFN-β in the media were measured using ELISA after one day. FIG. 3D demonstrates induction of IFN-β by purified mRNA. Vero cells were transfected with empty vector or plasmid containing L-II mut, or infected with wild-type PIV5, rPIV5VΔC, or mock-infected. mRNAs were purified and transfected into 293T cells, and IFN-β concentrations after one day were measured using ELISA. FIG. 3E demonstrates induction of IFN-β by L-II in the presence of CHX. 293T cells in 6-well plates were transfected with 1 μg of RNA or 250 ng of poly(I):poly©, and incubated with CHX (20 μg/ml) for 16 hours. The total RNAs were purified and subjected to reverse transcription and then real-time PCR analysis. ΔCT was calculated using actin from each sample as a control. FIG. 3F demonstrates lack of production of IFN-β in the absence of L-II mRNA. The purified L-II RNA was reverse transcribed using a L-II sequence-specific primer and a reverse transcriptase (RT). The product and/or purified L-II RNA were treated or untreated with RNase H (RH). The purified products were then transfected into 293T cells and IFN-β concentrations after one day were determined using ELISA. FIG. 3G demonstrates lack of production of IFN-β in the absence of the L mRNA. The same experiment as in FIG. 3F was carried out using RNAs purified from infected cells. RT(NP), reverse transcription using NP-specific primer; RT(L-II), reverse transcription using L-specific primer. The graph shows the average of three independent experiments and error bars represent SD. FIG. 3H demonstrates induction of IFN-β production by in vitro transcribed L-II RNA. The L-I and L-II RNA were in vitro synthesized using Riboprobe in vitro transcription systems (Promega). The RNA transcripts were treated or untreated with CIP to remove 5′-triphosphate and transfected into 293T cells. At one day post-transfection, IFN-β concentrations in the medium were measured using ELISA.

FIGS. 4A to 4C. The role of RIG-I in activation of NF-κB and IFN-β by L-II RNA. FIG. 4A demonstrates the effect of IPS-1 DN on activation of NF-κB by L RNA. A dual luciferase experiment was performed, as previously described in FIG. 1, using IPS-1 DN with a Flag tag (500 ng/μl). An immunoblotting experiment was performed to examine the expression of IPS-1-DN using anti-FLAG and anti-β-actin antibody. All transfections were carried out in replicates of four and error bars represent SD. FIG. 4B demonstrates activation of NF-κB by the L-II RNA was independent of RIG-I. At 18-20 hours after transfection, a dual luciferase assay was performed using lysate from Huh7 or Huh7.5 cells (RIG-I defective due to a T to I mutation at amino acid residue 55) transfected with vector, L, L-I, L-II, or L-II mut. FIG. 4C demonstrates the effect of RIG-I DN on activation of NF-κB by the L-II RNA. A reporter gene assay was performed using a plasmid expressing RIG-I DN with a Flag tag (500 ng/μl) along with the plasmids indicated. An immunoblotting experiment was performed to examine the expression of RIG-I-DN using anti-FLAG and anti-β-actin antibody. All transfections were carried out in replicates of four and error bars represent SD.

FIGS. 5A and 5B. MDA5 played a critical role in activation of IFN-β by viral mRNA. FIG. 5A addressed the role of RIG-I and MDA5 in activation of the IFN-β promoter by viral mRNA. 293T cells were transfected with siRNA targeting RIG-I, MDA5, or with NT siRNA. 48 h post-transfection of siRNA, the cells were transfected with vector, L-II mut, or with poly(I):poly©, along with the luciferase reporter plasmids. Luciferase activity was measured at 18-20 h post-transfection. FIG. 5B addressed the role of RIG-I and MDA5 in induction of IFN-β production by viral mRNA. siRNA transfection was performed as described in FIG. 5A in 293T cells, and at 48 h post-transfection of siRNA, the cells were transfected with vector, L-II mut, or with poly(I):poly©. After 18-20 h, amounts of IFN-β in the medium were measured using ELISA. Expression levels of RIG-I, MDA5, and β-actin were examined by immunoblotting.

FIGS. 6A to 6C. RNase L played a critical role in the activation of NF-B and IFN-β by viral mRNA. FIG. 6A addressed the role of RNase L in activating the IFN-β promoter. A dual luciferase assay for IFN-β promoter activation was performed as described in FIG. 3, using WT or RLKO (RNase L-deficient) MEFs. FIG. 6B demonstrates restoration of IFN-β activation in RLKO MEFs. IFN-β activation after complementing with RNase L cDNA in RLKO MEFs was examined by a dual luciferase experiment. RLKO were transiently transfected with RNase L cDNA or inactive RNase L mutant (R667A) cDNA. At 18 h after transfection, the cells were transfected with 1 g/1 of vector, L, L-I, L-I-II, or L-II mut plasmids, along with reporter plasmids. At one day post-transfection, the luciferase assay was performed. Amounts of RNase L and β-actin were examined by immunoblotting. All transfections were carried out in replicates of four and error bars represent SD. FIG. 6C demonstrates the role of RNase L in activating IFN-β expression. 293T cells were transfected with siRNA targeting RIG-I, MDA5, RNase L, or control siRNA. IFN-β production in response to vector, L-II mut, or poly(I):poly© was measured using ELISA. The expression of RIG-I, MDA5, and RNase L was examined by immunoblotting, with β-actin as loading control.

FIGS. 7A and 7B. Activation of NF-κB by region II of the L gene in an AKT-independent manner. FIG. 7A demonstrates activation of NF-κB by the L-II region of the L gene. BSR-T7 cells were transfected with a plasmid encoding a firefly luciferase gene (F-Luc) under the control of NF-κB-responsive elements, and increasing amounts (0, 500, 1000, 1500 ng) of a plasmid encoding PIV5 L, L-I, L-II, or L-I-II proteins, along with a plasmid encoding a Renilla luciferase (R-Luc) as an indicator of transfection efficiency. Empty vector was used to maintain a constant total of transfected DNA. Luciferase activities were measured at one day post-transfection. Ratios of F-Luc to R-Luc are used as an indicator of reporter gene activity. These ratios were normalized to the activity of the vector alone. All transfections were carried out in replicates of four and error bars represent standard deviation (SD). All P values were calculated using paired t test and shown in the figure. FIG. 7B demonstrates domain I of L is important for interaction with AKT1. ³⁵S-labeled L-I and L-II were synthesized by in vitro transcription and translation. AKT1 was obtained from cells transfected with an AKT1 expression plasmid. ³⁵S-labeled L-I or L-II was mixed with cell lysate containing AKT1 and immunoprecipitated with anti-AKT1 antibody.

FIGS. 8A to 8C. L-II RNA activated NF-κB. FIG. 8A presents schematics of the plasmid expressing L-II mut RNA. The L-II region was amplified using PCR primers that add two copies of HA tags at the C-terminal end of the L-II region, and subcloned into the EcoRI and NheI sites of the vector pCAGGS (Niwa et al., 1991, Gene; 108:193-200). The size of the L-II RNA transcript is about 1,000 nucleotides (nt) without poly(A). FIG. 8B demonstrates expression of L-II by L-II mut. The cells were transfected with vector, L-I, L-II, L-I-II, or L-II mut, and immunoprecipitation was performed to analyze the expression levels of the protein. FIG. 8C demonstrates expression of the L RNA. The amount of L-II and L-II mut RNA were compared using Northern blot with anti-L-II antisense DIG-labeled RNA probe. Methylene blue staining (below) was used to indicate the total RNA levels of the samples. “Marker” indicates the DIG-labeled RNA molecular weight marker. FIG. 8D demonstrates L-II mut activates NF-κB. A gel shift experiment was performed as described in FIG. 7 using appropriate competitors.

FIGS. 9A to 9C. Activation of NF-κB and IFN-β by L mRNA. A mutant L gene with two in-frame stop codons 6 nts downstream of the L start codon was generated (L mut). FIG. 9A demonstrates activation of NF-κB by the L mut. The reporter gene assay was performed as described in FIG. 1. FIG. 9B demonstrates activation of IFN-β promoter by the L mut. A dual luciferase assay was performed as described in FIG. 3. FIG. 9C demonstrates expression of L mutants. The cells were transfected with vector, L, or L mut, and immunoblotting was performed to analyze the expression levels of the proteins.

FIGS. 10A and 10B. The size of T7 RNA transcripts. The T7 RNA transcripts made in FIG. 3H were analyzed. FIG. 10A presents results as an agarose gel. FIG. 10B presents results as Agilent Bioanalyzer. Size markers are indicated.

FIG. 11. The role of MDA5 in activating NF-κB by viral mRNA. The cells were transfected with siRNA targeting MDA5 or with control siRNA (NT, non-target siRNA). At 48 hours after siRNA transfection, the cells were transfected with plasmids encoding L, L-II, or L-II mut, along with reporter luciferase genes. Luciferase activities were measured at 24 hours after transfection. The amounts of MDA5 and β-actin in the lysates from the dual luciferase assay were examined by immunoblotting.

FIG. 12. The role of RNase L in activating NF-κB. A dual luciferase assay for NF-κB activation was performed as described in FIG. 1, using WT or RNase L-deficient (RLKO) MEFs.

FIG. 13. Activated IFN-β expression by the mRNA of MuV L.

FIG. 14. Role of MDA5 in the activation of IFN-β by viral mRNA of MuV L.

FIGS. 15A and 15B. Predicted structure of the RNA. FIG. 15A presents the entire RNA. Structure was predicted using RNAFOLD. The predicted stem-loop structure is circled. FIG. 15B presents the predicted stem-loop.

FIG. 16. Induction of IFN beta in mice following intramuscular injection of plasmid encoding mRNA (L-IImut), vector only plasmid, or PBS.

FIG. 17. The DNA (SEQ ID NO:1) and encoded amino acid sequence (SEQ ID NO:2) of the L-IImut transcript.

FIG. 18. Activation of NF-κB by mutations of region II of the L gene.

FIG. 19 shows the reduction of influenza virus by the L-II in vivo. 6- 8 weeks old female BALB/c mice (Harlan, Indianapolis, Ind.) in a group more than 5 were injected intramuscularly with 100 μl of DNA plasmid containing the L-II region of the L gene (2 μg/μl), empty vector (2 μg/μl), or sterile PBS. One day after injection with plasmid or PBS, mice were infected intranasally 100 μl of A/PR/8/34 (H1N1; 600 PFU). Naive mice inoculated with either virus were used as controls. The TCID₅₀ was determined for lungs harvested from influenza infected mice and the TCID₅₀ was calculated by the Reed and Meunch method.

### DETAILED DESCRIPTION OF ILLUSTRATIVE EMBODIMENTS OF THE PRESENT INVENTION

The present invention includes novel agents that are capable of activating the expression of interferon (IFN), a critical cytokine for preventing viral infection. These agents are stable, easy to produce, and have the potential to increase efficacy by specifically targeting diseased organs and tissues and reduce the side-effects associated with the systemic delivery of IFN. More specifically, these IFN activating agents include single stranded nucleotide sequences that induce the expression of interferon beta (IFN-β) through a RNase L and/or MDA5-dependent pathway. MDA5 (melanoma differentiation associated gene 5), a RNA helicase, plays an essential role in activation of IFN expression (Andrejeva et al., 2004, Proc Natl Acad Sci USA;101(49):17264- 17269). The recognition by MDA-5 of the natural RNAs generated during viral infections leads to activation of IPS-1 (interferon-beta promoter stimulator-1), NF-κB, and IFN expression (Kawai and Akira, 2006, Nat Immunol; 7(2):131-7). How MDA5 differentiates between self and non-self RNA is unclear. It has been reported that stable, long, double-stranded RNA (dsRNA) structures greater than 2 kilobase pairs (kb) in size, presumably with 5′-triphosphates, generated during RNA virus infection (not typical of self RNA), serve as a distinguishing factor for MDA5-specific recognition (Kato et al., 2008, J Exp Med; 205(7):1601-10). And while long, synthetic, double stranded (ds) RNA polymers of poly(I):poly(C) are often used as activators of MDA5 (Gitlin et al., 2006, Proc Natl Acad Sci USA; 103(22):8459-64), the present invention provides the first demonstration of the activation of IFN expression through MDA5 by single-stranded RNAs (ssRNA).

The immune system has evolved to recognize pathogens via pathogen recognition receptors and pathogen associated molecular patterns. This innate immunity plays a critical role in host defense against a variety of pathogens, including viral infections. The recognition of pathogen associated molecular patterns results in the rapid induction of antiviral cytokines, including IFN. Such an innate immune response mediated by interferons (IFNs) is a front line defense against viral infections in vertebrate animals.

The IFN activating agents described herein may activate an innate immune response. The induction of innate immune responses requires activation of transcription factors. In particular, NF-κB plays an essential role in activating the expression of cytokines involved in innate immunity, such as interferon beta (IFN-β) or interleukin-6 (IL-6). The IFN activating agents described herein may activate NF-κB expression. In addition to activating expression of IFN-β, an IFN activating agent may activate the expression of other cytokines, such as, for example, IL-1, IL-1, IL-2, IL-3, IL-4, IL-6, IL-8, IL-9, IL-10, IL-12, IL-13, IL-15, IL-18, IL-19, IL-20, IFN-α, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF), and/or Flt-3 ligand. In addition to activating expression of IFN-β, an IFN activating agent may activate the expression of other interferons, such as, for example, interferon alpha (IFN-α) and/or interferon gamma (IFN-γ).

An IFN activating agent may be a single stranded polynucleotide sequence or a transcription or expression vector that provides for the transcription of such a single stranded polynucleotide sequence. In preferred embodiments, an IFN activating agent is not a double stranded polynucleotide sequence or a polypeptide. An IFN activating agent may demonstrate one or more of the functional effects described herein. For example, an IFN activating agent may activate expression of IFN-β, activate NF-κB expression, activate an innate immune response, activate the expression of one or more cytokines, and/or induce the expression of interferon beta (IFN-β) through a RNase L and/or MDA5-dependent pathway.

A single stranded polynucleotide sequence may be a single stranded ribonucleic acid (RNA) sequence. Such a single stranded RNA sequence may be, for example, a messenger RNA (mRNA) or a non-coding RNA. In some embodiments, a single stranded RNA may have, for example, a 5′ cap and/or a poly(A) tail. In some embodiments, a single stranded RNA may lack, for example, a 5′ cap and/or a poly(A) tail. In some embodiments, a single stranded RNA sequence is not translated into an amino acid sequence, including, for example, including one or more stop codons that prevent translation. Such a stop codon may be located, for example, in the 5′ portion of the sequence. Such a stop codon may be positioned so that it replaces a start codon. In the genetic code, a stop codon (or termination codon) is a nucleotide triplet within messenger RNA that does not code for an amino acid and signals a termination of translation, thus signaling the end of protein synthesis. In the standard genetic code, stop codons include the RNA sequences UAG, UAA, and UGA, and the DNA sequences TAG, TAA, and TGA. In the standard genetic code, a start codon includes the RNA sequence AUG and the DNA sequence ATG. A single stranded polynucleotide sequence may be a deoxyribnucleic acid (DNA) sequence including, but not limited to, a DNA sequence that corresponds to a single stranded RNA as described herein (for example, with thymine (T) residues rather than uracil (U) residues), or a DNA sequence that transcribes a single stranded RNA sequence, as described herein.

An IFN activating agent may be a single stranded nucleotide sequence, including, but not limited to, a single stranded RNA sequence, that includes a nucleotide sequence encoding conserved region II of the L protein of a negative-stranded RNA virus, or a fragment of the conserved region II of a L protein. Also include are single stranded nucleotide sequences including a sequence that is about 70%, about 75%, about 80%, about 85%, about 90%, about 91%, about 92%, about 93%, about 94%, about 95%, about 96%, about 97%, about 98%, or about 99% identical to a nucleotide sequence encoding conserved region II of the L protein of a negative stranded RNA virus, or a fragment thereof. Also include are single stranded nucleotide sequences including a sequence that encodes an amino acid sequence that is about 70%, about 75%, about 80%, about 85%, about 90%, about 91%, about 92%, about 93%, about 94%, about 95%, about 96%, about 97%, about 98%, or about 99% identical to a the amino acid sequence of conserved region II of the L protein of a negative stranded RNA virus, or a fragment thereof. Such an IFN activating agent may demonstrate one or more of the functional effects described herein. For example, an IFN activating agent may activate expression of IFN-β, activate NF-κB expression, activate an innate immune response, activate the expression of one or more cytokines, and/or induce the expression of interferon beta (IFN-β) through a RNase L and/or MDA5-dependent pathway. In some embodiments, the single stranded nucleotide sequence does not include sequence that encode conserved region I, III, IV, V, and/or VI of the L protein. In some embodiments, a single stranded nucleotide sequence is not translated into an amino acid sequence, including, for example, with one or more stop codons that prevent translation. Such a stop codon may be located, for example, in the 5′ portion of the sequence. Such a stop codon may be positioned so that it replaces a start codon.

An IFN activating agent may be a single stranded nucleotide sequence, including, but not limited to, a single stranded RNA sequence, that hybridizes to a nucleotide sequence encoding conserved region II of the L protein of a negative stranded nonsegmented RNA virus, or a fragment of the conserved region II of a L protein. Such hybridization includes moderate stringency hybridization conditions or high stringency hybridization conditions. High stringency conditions may be, for example, 6×SSC, 5× Denhardt, 0.5% sodium dodecyl sulfate (SDS), and 100 μg/ml fragmented and denatured salmon sperm DNA hybridized overnight at 65° C. and washed in 2×SSC, 0.1% SDS at least one time at room temperature for about 10 minutes followed by at least one wash at 65° C. for about 15 minutes followed by at least one wash in 0.2×SSC, 0.1% SDS at room temperature for at least 3 to 5 minutes. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like. Such an IFN activating agent may demonstrate one or more of the functional effects described herein. For example, an IFN activating agent may activate expression of IFN-β, activate NF-κB expression, activate an innate immune response, activate the expression of one or more cytokines, and/or induce the expression of interferon beta (IFN-β) through a RNase L and/or MDA5-dependent pathway. In some embodiments, the single stranded nucleotide sequence does not include sequence that encode conserved region I, III, IV, V, and/or VI of the L protein. In some embodiments, a single stranded nucleotide sequence is not translated into an amino acid sequence, including, for example, with one or more stop codons that prevent translation. Such a stop codon may be located, for example, in the 5′ portion of the sequence. Such a stop codon may be positioned so that it replaces a start codon.

Negative stranded RNA viruses have a linear genome that is a single stranded minus sense, negative polarity RNA. The genome encodes an RNA-directed RNA polymerase (L), hemagglutinin-neuraminidase protein (HN), fusion protein (F), matrix protein (M), phosphoprotein (P) and nucleoprotein (N) in the 5-3 direction. Negative stranded RNA viruses include, but are not limited to, viruses of the Paramyxoviridae family, including the Paramyxovirinae and Pneumovirinae subfamilies. Negative-sense single-stranded RNA viruses of the Paramyxoviridae family are responsible for a number of human and animal diseases. Examples of viruses of the viruses of the Paramyxoviridae family, include, but are not limited to, human parainfluenza virus 1, human parainfluenza virus 2, human parainfluenza virus 3, human parainfluenza virus 4, parainfluenza virus 5, mumps virus, measles virus, human metapneumovirus, human respiratory syncytial virus, bovine respiratory syncytial virus rinderpest virus, canine distemper virus, phocine distemper virus, cetacean morbillivirus, Newcastle disease virus, avian pneumovirus, Peste des Petits Ruminants virus (PPRV), Sendai virus, Menangle virus, Tupaia paramyxovirus, Tioman virus, Tuhokovirus 1, Tuhokovirus 2, Tuhokovirus 3, Hendra virus, Nipah virus, Fer-de-Lance virus, Nariva virus, Salem virus, J virus, Mossman virus, and Beilong virus. Other negative-sense single-stranded RNA viruses include, for example, members of the Rhabdoviridae family, including, but not limited to, vesicular stomatitis virus and rabies virus.

The L genes from a number of Paramyxoviridae have been sequenced, and all are about the same size, approximately 2200 amino acids. There are six highly conserved blocks of amino acids among RNA-dependent RNA polymerases from diverse viral families, and these may be regions essential for the enzymatic polymerase activity. The full length genomic sequences and amino acid sequences for the L protein of many negative stranded RNA viruses are readily available. For example, genomic and amino acid sequences for the L protein of many members of the Paramyxoviridae family are available in GenBank, including, but not limited to, avian paramyxovirus serotype 6 (NC 003043), bovine parainfluenza virus 3 (NC 002161), bovine respiratory syncytial virus (NC 001989), canine distemper virus (NC 001921), Hendra virus (NC 001906), human metapneumovirus (NC 004148), human parainfluenza virus 1 (NC 003461), human parainfluenza virus 2 (NC 003443), human parainfluenza virus 3 (NC 001796), human respiratory syncytial virus (NC 001781), measles virus (NC 001498), mumps virus (NC 002200), Nipah virus (NC 002728), rinderpest virus (X98291), Sendai virus (NC 001552), simian parainfluenza virus 41 (X64275), simian parainfluenza virus 5 (AF052755), Tioman virus (NC 004074), Tupaia virus (NC 002199), and the avian metapneumovirus (strain Colorado) L gene (AY394492). See, for example, Poch et al., 1990, J Gen Virol; 71 (Pt 5):1153-62; Sidu et al., 1993, Virology; 193(1):50-65; and Wise et al., 2004, Virus Research; 104:71-80). An activating agent may include the sequence of conserved region II of the L protein from one or more of these sequences. In some embodiments, the single stranded nucleotide sequence does not include sequence that encode conserved region I, III, IV, V, and/or VI of the L protein.

An IFN activating agent may be a single stranded nucleotide sequence, including, but not limited to, a single stranded RNA sequence, that includes a nucleotide sequence that is about 70%, about 75%, about 80%, about 85%, about 90%, about 91%, about 92%, about 93%, about 94%, about 95%, about 96%, about 97%, about 98%, or about 99% identical to SEQ ID NO:1 (nucleotide sequence encoding conserved region II of the L protein of the parainfluenza virus 5), or a fragment thereof. An IFN activating agent may be a single stranded nucleotide sequence that includes a nucleotide sequence that hybridizes to SEQ ID NO:1, or a fragment thereof, under high stringency or moderate stringency, or a complement thereof. An IFN activating agent may be a single stranded nucleotide sequence that includes SEQ ID NO:1, or a fragment thereof. Such an IFN activating agent may demonstrate one or more of the functional effects described herein. For example, an IFN activating agent may activate expression of IFN-β, activate NF-κB expression, activate an innate immune response, activate the expression of one or more cytokines, and/or induce the expression of interferon beta (IFN-β) through a RNase L and/or MDA5-dependent pathway. In some embodiments, the single stranded nucleotide sequence does not include sequence that encode conserved region I, III, IV, V, and/or VI of the L protein. In some embodiments, a single stranded nucleotide sequence is not translated into an amino acid sequence, including, for example, with one or more stop codons that prevent translation. Such a stop codon may be located, for example, in the 5′ portion of the sequence. Such a stop codon may be positioned so that it replaces a start codon.

An IFN activating agent may be a single stranded nucleotide sequence including a sequence that encodes an amino acid sequence that is about 70%, about 75%, about 80%, about 85%, 90%, about 91%, about 92%, about 93%, about 94%, about 95%, about 96%, about 97%, about 98%, or about 99% identical to a the amino acid sequence SEQ ID NO:2 (amino acid sequence of conserved region II of the L protein of the parainfluenza virus 5), or a fragment thereof. An IFN activating agent may be a single stranded nucleotide sequence including a sequence that encodes the amino acid sequence of SEQ ID NO:2, or a fragment thereof. Such an IFN activating agent may demonstrate one or more of the functional effects described herein. For example, an IFN activating agent may activate expression of IFN-β, activate NF-κB expression, activate an innate immune response, activate the expression of one or more cytokines, and/or induce the expression of interferon beta (IFN-β) through a RNase L and/or MDA5-dependent pathway. In some embodiments, the single stranded nucleotide sequence does not include sequence that encode conserved region I, III, IV, V, and/or VI of the L protein. In some embodiments, a single stranded nucleotide sequence is not translated into an amino acid sequence, including, for example, with one or more stop codons that prevent translation. Such a stop codon may be located, for example, in the 5′ portion of the sequence. Such a stop codon may be positioned so that it replaces a start codon.

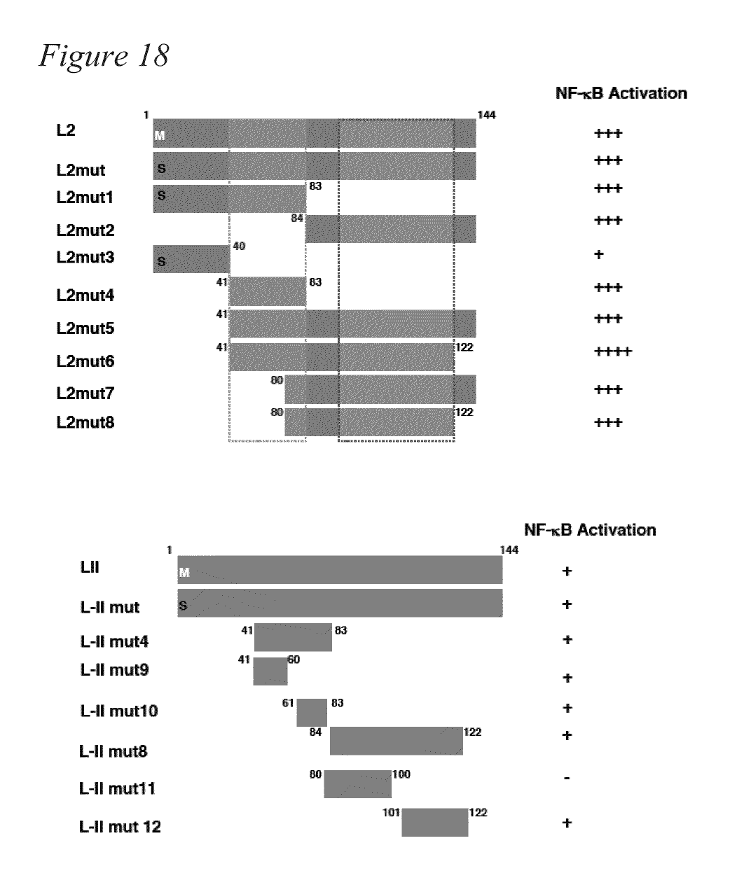

An IFN activating agent may be a single stranded nucleotide sequence that includes a nucleotide sequence encoding a fragment of the amino acid sequence of the conserved region II of a L protein of a negative stranded RNA virus, including, but not limited to, a fragment of the amino acid sequence of the conserved region II of a L protein of a virus of the Paramyxoviridae family. Such a fragment may include, for example, about amino acid 500 to about amino acid 600, about amino acid 503 to about amino acid 600, about amino acids 615 to about amino acids 640, or about amino acid 580 to about amino acid 680 of the L protein. Fragments include any of those show in FIG. 18. A fragment may exclude about the first 40 amino acids of region II of the L protein. A fragment may include about amino acids 40 to about 80, amino acids about 40 to about 100, amino acids about 40 to about 120, amino acids about 40 to about 140, amino acids about 80 to about 100, amino acids about 80 to about 120, and amino acids about 80 to about 140 of region II of the L protein.

Fragments include, but are not limited to, for example, fragments having about 5, about 10, about 15, about 20, about 25, about 50, about 75, about 100, about 150, about 200, about 250, about 300, about 350, about 400, about 450, about 500, about 550, about 600, about 650, or about 700 consecutive nucleotides of a sequence encoding an L protein of a negative stranded RNA virus, including, but not limited to, a virus of the Paramyxoviridae family, or encoding conserved region II of a L protein negative stranded RNA virus, including, but not limited to, viruses of the Paramyxoviridae family Fragments include, but are not limited to, for example, fragments having about 5, about 10, about 15, about 20, about 25, about 50, about 75, about 100, about 150, about 200, about 250, about 300, about 350, about 400, about 450, about 500, about 550, about 600, about 650, or about 700 consecutive amino acids of an L protein of a negative stranded RNA virus, including, but not limited to, a virus of the Paramyxoviridae family, or of the conserved region II of a L protein negative stranded RNA virus, including, but not limited to, viruses of the Paramyxoviridae family, and nucleotide sequences encoding such amino acid fragments.

A fragment may include a portion of region II that is highly conserved the between negative stranded RNA viruses. A fragment may include a portion of region II that is highly conserved the between viruses of the Paramyxoviridae family. In some embodiments, a fragment does not include sequences that encode conserved region I, III, IV, V, and/or VI of the L protein. In some embodiments, the single stranded nucleotide sequence of a fragment is not translated into an amino acid sequence, including, for example, with one or more stop codons that prevent translation. Such a stop codon may be located, for example, in the 5′ portion of the sequence. Such a stop codon may be positioned so that it replaces a start codon. Such a fragment may demonstrate one or more of the functional effects described herein. For example, activating expression of IFN-β, activating NF-κB expression, activating an innate immune response, activating the expression of one or more cytokines, and/or inducing the expression of interferon beta (IFN-β) through a RNase L and/or MDA5-dependent pathway.

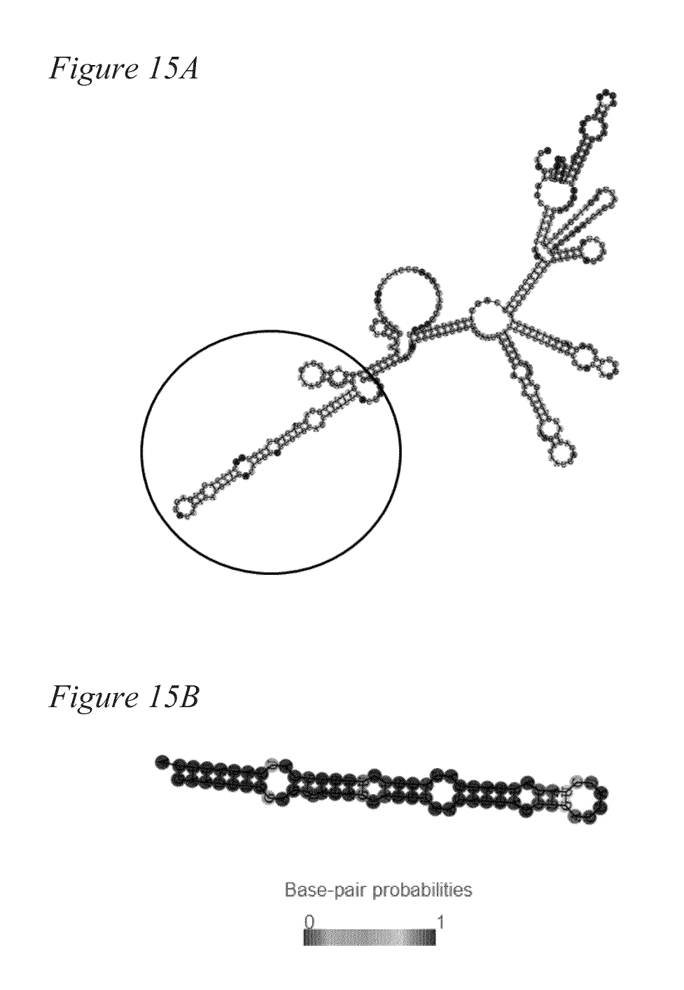

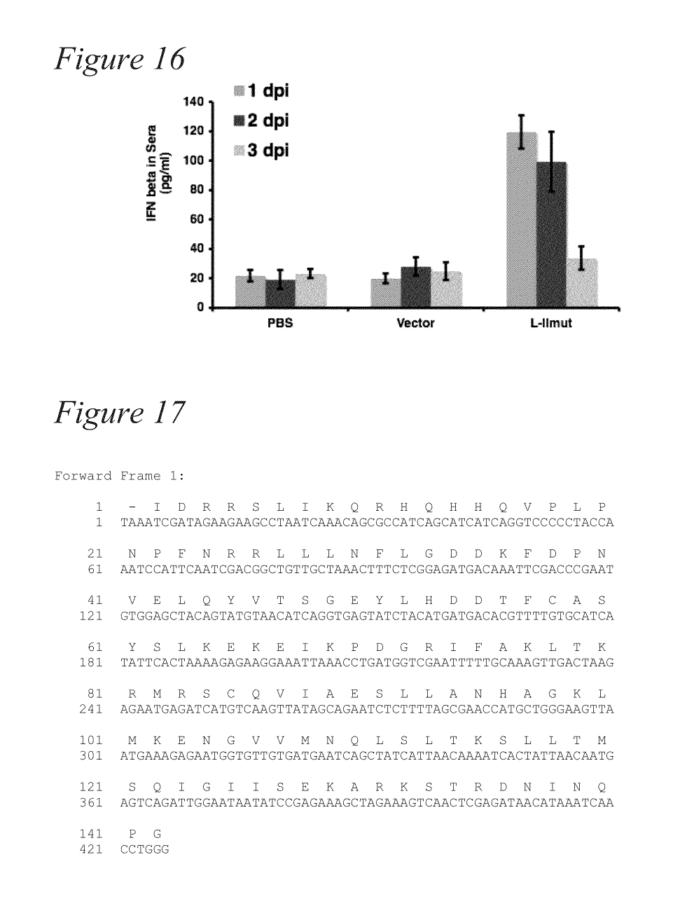

An IFN activating agent may be a single stranded nucleotide sequence that is the result of RNase H processing of a single stranded nucleotide sequence (either a RNA or DNA) encoding the amino acid sequence of the conserved region II of a L protein of a negative stranded RNA virus, including, but not limited to, a fragment of the amino acid sequence of the conserved region II of a L protein of a virus of the Paramyxoviridae family. Such processing may take place under conditions that allow base pair hybridization and/or stem/loop formation (for example, as shown in FIG. 15A) in a nucleotide sequence encoding an amino acid sequence of conserved region II of a L protein. The enzyme RNase H is a non-specific endonuclease and catalyzes the cleavage of RNA. RNase H′s ribonuclease activity cleaves the 3′-O—P bond of RNA in a DNA/RNA duplex to produce 3′-hydroxyl and 5′-phosphate terminated products. In DNA replication, RNase H is responsible for removing the RNA primer, allowing completion of the newly synthesized DNA. RNase H specifically degrades the RNA in RNA:DNA hybrids and will not degrade DNA or unhybridized RNA.

An IFN activating agent may be a transcription or expression vector that includes sequences that provide for the transcription of a single stranded nucleotide sequence as described herein. In preferred embodiments, the transcription or expression vector includes sequence that provide for the transcription of a single stranded RNA sequence as described herein. Such vectors include any of a wide variety of plasmid or viral vectors, including, but not limited to an of those described herein. Such a vector may include a tissue specific or inducible promoter.

The IFN activating agents described herein may be used in vitro, ex vivo, and/or in vivo for the activation of the expression of IFN-β or other cytokines, the activation of a RNase L and/or MDA5-dependent pathway, and/or the activation of NF-κB expression. As used herein in vitro is in cell culture, ex vivo is a cell that has been removed from the body of a subject, and in vivo is within the body of a subject. As used herein, the term “activate” means induce and/or increase.

The IFN activating agents described herein may be administered to a subject for the treatment and/or prevention of viral diseases, infections, cancer, autoimmune diseases, chronic conditions, and other diseases in which the administration of IFN-β, activation of the expression of IFN-β or other cytokines, activation of a RNase L and/or MDA5-dependent pathway, and/or activation of NF-κB expression is therapeutically advantageous. As used herein “treating” or “treatment” includes therapeutic and/or prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. An IFN activating agent may be administered to a subject prior to and/or after exposure to a virus or infectious agent. As used herein, the term “subject” or “individual” represents an organism, including, for example, a mammal. A mammal includes, but is not limited to, a human, a non-human primate, livestock (such as, but not limited to, a cow, a horse, a goat, and a pig), a rodent, such as, but not limited to, a rat or a mouse, or a domestic pet, such as, but not limited to, a dog or a cat.

With the methods of the present invention, an IFN activating agent as described herein may be directly administered to a subject. Or, in preferred embodiments, an IFN activating agent may be delivered to a subject by a transcription or expression vector that includes sequences that provide for the transcription of a single stranded nucleotide sequence as described herein. In preferred embodiments, the transcription or expression vector includes sequences that provide for the transcription of a single stranded RNA sequence as described herein. Such vectors include any of a wide variety of plasmid or viral vectors, including, but not limited to an of those described herein. Such a vector may include an organ or tissue specific promoter or an inducible promoter.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

An IFN activating agent as described herein may be administered to a patient to inhibit and/or prevent the replication of a virus. An IFN activating agent as described herein may be administered to a patient for the treatment or prevention of a viral infection. Such viral infections include, for example, hepatitis, such as for example, hepatitis C, hepatitis B, or hepatitis A, influenza, such as, for example, influenza A (including, but not limited to, the H5N1 and H1N1 subtypes), influenza B, and influenza C, respiratory syncytial virus (RSV), and rabies. Such viral infections include, for example, infection with a negative stranded RNA virus, such as for example, a virus of the family Paramyxoviridae. Examples of a virus of the family Paramyxoviridae include, but are not limited to, human parainfluenza virus 1, human parainfluenza virus 2, human parainfluenza virus 3, human parainfluenza virus 4, parainfluenza virus 5, mumps virus, measles virus, human metapneumovirus, human respiratory syncytial virus, bovine respiratory syncytial virus rinderpest virus, canine distemper virus, phocine distemper virus, Newcastle disease virus, avian pneumovirus, Peste des Petits Ruminants virus (PPRV), Sendai virus, Menangle virus, Tupaia paramyxovirus, Tioman virus, Tuhokovirus 1, Tuhokovirus 2, Tuhokovirus 3, Hendravirus, Nipahvirus, Fer-de-Lance virus, Nariva virus, Salem virus, J virus, Mossman virus, and Beilong virus. In some aspects, an IFN activating agent as described herein may be administered to a patient for the treatment or prevention of a other infectious diseases, including, but not limited to bacterial, fungal and parasitic infections.

The methods of the present disclosure may be administered to a patient for the treatment of cancer. Cancers to be treated include, but are not limited to, melanoma, basal cell carcinoma, colorectal cancer, pancreatic cancer, breast cancer, prostate cancer, lung cancer (including small-cell lung carcinoma and non-small-cell carcinoma), leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, glioblastoma, and adrenal cortical cancer. In some aspects, the cancer is cancer for which the administration of IFN is effective. In some aspects, the cancer is a melanoma. In some aspects, the cancer is a primary cancer. In some aspects, the cancer is metastatic, including, but not limited to a metastatic melanoma.

The efficacy of treatment of a cancer may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, determinations of tumor infiltrations by immune system cells, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation.

The methods of the present disclosure may be administered to a patient for the treatment of an autoimmune disease, such as, for example, multiple sclerosis (MS), systemic lupus erythematosus (SLE), and rheumatoid arthritis (RA). The methods of the present disclosure may be administered to a patient for the treatment of a neurological disorder, such as, for example, multiple sclerosis (MS).

Also included in the present invention are compositions including one or more of the IFN activating agents described herein. Such a composition may include pharmaceutically acceptable carriers or diluents. Carriers include, for example, stabilizers, preservatives and buffers. Suitable stabilizers include, for example, SPGA, carbohydrates (such as sorbitol, mannitol, starch, sucrose, dextran, glutamate or glucose), proteins (such as dried milk serum, albumin or casein) or degradation products thereof. Suitable buffers include, for example, alkali metal phosphates. Suitable preservatives include, for example, thimerosal, merthiolate and gentamicin. Diluents, include, but are not limited to, water, aqueous buffer (such as buffered saline), alcohols, and polyols (such as glycerol).

An IFN activating agent as described herein may be administered once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

By a “therapeutically effective amount” is meant a sufficient amount of the compound to treat the subject at a reasonable benefit/risk ratio applicable to obtain a desired therapeutic response. It will be understood, however, that the total daily usage of the compounds and compositions of the present invention will be decided by the attending physician within the scope of sound medical judgment. The specific therapeutically effective dose level for any particular patient will depend upon a variety of factors including, for example, the disorder being treated and the severity of the disorder, activity of the specific compound employed, the specific composition employed, the age, body weight, general health, sex and diet of the patient, the time of administration, route of administration, and rate of excretion of the specific compound employed, the duration of the treatment, drugs used in combination or coincidentally with the specific compound employed, and like factors well known in the medical arts.

IFN activating agents, as described herein, can be administered by any suitable means including, but not limited to, for example, parenteral (involving piercing the skin or mucous membrane), oral (through the digestive tract), transmucosal, rectal, nasal, topical (including, for example, transdermal, aerosol, buccal and sublingual), or vaginal. Parenteral administration may include, for example, subcutaneous, intramuscular, intravenous, intradermal, intraperitoneal, intrasternal, and intraarticular injections as well as various infusion techniques.

IFN activating agents, as described herein, can be administered in a localized fashion, rather than systemically. For example, IFN activating agents may be formulated for aerosol or inhalation administration to the lungs, respiratory tract, and/or mucosal membranes. Such formulations may be especially effective for the treatment and prevention of viral infections acquired by respiratory exposure. Hepatic delivery of IFN activating agents may be especially effective for the treatment of hepatitis C and liver cancer, including, but not limited to, metastatic cancer of the liver. IFN activating agents may also be delivered locally by means including, but not limited to, intramuscular, subcutaneous, topical, intraocular, and intratumor. Localized deliver may be to any of a variety of tissues or organs, such as, for example, lung, liver, muscle, nervous tissue, central nervous system, brain, spinal cord, skin, or tumor. In some embodiments, when localized delivery is effected by a plasmid, the plasmid may also include an inducible promoter or tissue specific promoter, such as for example, a liver specific promoter.

For human and veterinary administration, IFN activating agents, as described herein, may meet sterility, pyrogenicity, and general safety and purity standards as required by the FDA. Such compositions are considered suitable for parenteral or enteral administration to a mammal Such compositions may be pyrogen-free.

Compositions may be administered in any of the methods of the present invention and may be formulated in a variety of forms adapted to the chosen route of administration. The formulations may be conveniently presented in unit dosage form and may be prepared by methods well known in the art of pharmacy. A composition may include a pharmaceutically acceptable carrier. The term “pharmaceutically acceptable,” as used herein, means that the compositions or components thereof so described are suitable for use in contact with human skin without undue toxicity, incompatibility, instability, allergic response, and the like. A composition may be a pharmaceutical composition.

As used herein, the term isolated means a preparation that is either removed from its natural environment or synthetically derived, for instance by recombinant techniques, or chemically or enzymatically synthesized.

In accordance with the present invention, an IFN activating agents may be administered in combination with the administration of one or more previously known treatment modalities. As used herein, the term “additional therapeutic agent” represents one or more agents previously known to be therapeutically effective. In some embodiments, such an additional therapeutic agent is not a single stranded RNA. The administration of an IFN activating agent may take place before, during, and/or after the administration of the other mode of therapy. The present invention includes methods of administering one or more IFN activating agents in combination with the administration of one or more previously known treatment modalities. The present invention includes compositions of one or more IFN activating agents and one or more previously known treatment modalities.

In some embodiments of the present invention, the administration of an IFN activating agent in combination with additional therapeutic agents may demonstrate therapeutic synergy. Likewise, the administration of two or more IFN activating agents may demonstrate therapeutic synergy. As used herein, a combination may demonstrate therapeutic synergy if it is therapeutically superior to one or other of the constituents used at its optimum dose (Corbett et al., 1982, Cancer Treatment Reports; 66:1187. In some embodiments, a combination demonstrates therapeutic synergy if the efficacy of a combination is characterized as more than additive actions of each constituent.

The IFN activating agents described herein may be used to provide adjuvant activity in methods of inducing an immune response and immunization methods. The present invention includes immunogenic compositions that include, as one aspect, one or more antigenic agents, and as a second aspect, one or more of the IFN activating agents as described herein. Such compositions may include a single stranded IFN activating agent as described. Alternatively, such compositions may include a transcription or expression vector including sequences that provide for the transcription of a single stranded IFN activating agent. Such vectors include any of a wide variety of plasmid or viral vectors, including, but not limited to an of those described herein. Such compositions may be used as vaccines.

The one or more antigenic agent may be any of the great variety of antigens that are administered to a subject to elicit an immune response in the subject. An antigenic aspect may be an immunogen derived from a pathogen. The antigenic aspect may be, for example, a peptide antigen, a protein antigen, a viral antigen or polypeptide, an inactivated virus, a bacterial or parasitic antigen, an inactivated bacteria or parasite, a whole cell, a genetically modified cell, a tumor associated antigen or tumor cell, or a carbohydrate antigen. In some embodiments, the antigenic agent is a DNA vaccine, that is, the antigenic agent is delivered as a vector construct, such as a plasmid, that results in the expression of a polypeptide antigen upon delivery to a subject. When the antigenic aspect is a DNA vaccine, the IFN activating agent may be provided as a separate component. Or, in some embodiments, the antigenic agent and the IFN activating agent are delivered in a single vector construct. The present invention includes such vector constructs.

The present invention also includes methods for the administration of such compositions to a subject to elicit an immune response in the subject. The immune response may or may not confer protective immunity. An immune response may include, for example, a humoral response and/or a cell mediated response. Such an immune response may result in a reduction or mitigation of the symptoms of future infection. Such an immune response may prevent a future infection. Such an immune response may prevent a cancer. Such an immune response may result in the reduction of symptoms of a cancer. Such an immune response may treat a cancer. Such an immune response may be a humoral immune response, a cellular immune response, and/or a mucosal immune response. A humoral immune response may include an IgG, IgM, IgA, IgD, and/or IgE response. The determination of a humoral, cellular, or mucosal immune response may be determined by any of a variety of methods, including, but not limited to, any of those described herein. The induction of an immune response may include the priming and/or the stimulation of the immune system to a future challenge with an infectious agent or cancer, providing immunity to future infections or cancers. The induction of such an immune response may serve as a protective response, generally resulting in a reduction of the symptoms.

The present invention includes kits including one or more IFN activating agents as described herein. Such kits may further include one or more antigenic agents, as described herein. A kit may include appropriate negative controls and/or a positive controls. Kits of the present invention may include other reagents such as suitable buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well known methods, preferably to provide a sterile, contaminant-free environment. As used herein, the term “package” refers to a solid matrix or material such as glass, plastic, paper, foil, and the like, capable of holding within fixed limits a polypeptide. Kits of the present invention may also include instructions for use. Instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Activation of Interferon-β Expression by a Viral mRNA through RNase L and MDA5

Interferons (IFNs) play a critical role in innate immunity against viral infections. Melanoma Differentiation-Associated Protein 5 (MDA5), a RNA helicase, is a key component in activating expression of type I IFNs in response to certain types of viral infection. MDA5 senses non-cellular RNA and triggers the signaling cascade that leads to IFN production. Synthetic double-stranded RNAs are known activators of MDA5. However, natural single-stranded RNAs have not been reported to activate MDA5. This example identified a viral mRNA from parainfluenza virus 5 (PIV5) that activates IFN expression through MDA5 and provide evidence that the signaling pathway includes the antiviral enzyme, RNase L. The L mRNA of PIV5 activated expression of IFN-β. This RNA was mapped to a region of 430 nucleotides within the L mRNA of PIV5. This example indicates that a viral mRNA, with 5′-cap and 3′-poly (A), can activate IFN expression through a RNase L-MDA5 pathway.

### Introduction

Interferons (IFN) play a critical role in innate immune responses to viral infections. Viruses trigger expression of IFN-β in infected cells, and IFN-β can lead to activation of IFN-α expression through phosphorylation of IRF-7 (interferon regulatory factor-7) (Marie et al., 1998, EMBO J; 17(22):6660-9; and Sato et al., 1998, FEBS Lett; 441(1):106-10). IFNs induce an antiviral state in cells that inhibits the spread of infection. MDA5 (melanoma differentiation associated gene 5), a RNA helicase, plays an essential role in activation of IFN expression (Andrejeva et al., 2004, Proc Natl Acad Sci USA; 101(49):17264-17269). MDA5 is involved in cytoplasmic sensing of infections by some RNA viruses (Akira et al., 2006, Cell; 124(4):783-801). Recognition of RNA molecules generated during viral infections by MDA5 leads to activation of IPS-1 (interferon-beta promoter stimulator-1), NF-κB, and IFN expression (Kawai and Akira, 2006, Nat Immunol; 7(2):131-7). How MDA5 differentiates between self and non-self RNA is unclear. It has been reported that stable, long, double- stranded (ds)RNA structures greater than 2 kilobase pairs (kb) in size, presumably with 5′-triphosphates, generated during RNA virus infection (not typical of self RNA), may serve as a distinguishing factor for MDA5-specific recognition (Kato et al., 2008, J Exp Med; 205(7):1601-10). Long, synthetic, dsRNA polymers of poly(I):poly© are often used as a surrogate for the putative activator of MDA5 (Gitlin et al., 2006, Proc Natl Acad Sci USA; 103(22):8459-64). A natural single-stranded (ss)RNA trigger for MDA5 has not been identified.

The role of MDA5 in regulating interferon (IFN) expression was first reported in studies of parainfluenza virus 5 (PIV5) (formerly known as simian virus 5 (SV5)) (Andrejeva et al., 2004, Proc Natl Acad Sci USA; 101(49):17264-17269; and Chatziandreou et al., 2004, J Gen Virol; 85(Pt 10):3007-16). PIV5 is a prototypical paramyxovirus in a family of non-segmented, negative-stranded RNA viruses, which includes many important human and animal pathogens, such as mumps virus, measles virus, Nipah virus, and respiratory syncytial virus (Lamb and Kolakofsky, Paramyxoviridae: The viruses and their replication, in Fields Virology (Fourth Edition), D. M. Knipe and P. M. Howley, Editors. 2001, Lippincott, Williams and Wilkins: Philadelphia). The viral RNA-dependent RNA polymerase (vRdRp), minimally consisting of the L protein and the P protein, transcribes the nucleocapsid protein (NP or N)-encapsidated viral genome RNA into 5′ capped and 3′ polyadenylated mRNAs (Emerson et al., 1975, J. Virol; 15:1348-1356).

The V protein of PIV5, a component of PIV5 virions (˜350 molecules per virion), is a multifunctional protein and plays important roles in viral pathogenesis. The V protein C-terminal domain contains seven cysteine residues, resembling a zinc finger domain, and binds atomic zinc (Paterson et al., 1995, Virology; 208:121-131). A recombinant virus lacking the C-terminus of the V protein of PIV5 (rPIV5VΔC) induces a higher level of IFN expression than wild-type virus, indicating that the V protein plays an essential role in blocking IFN production in virus-infected cells (He et al., 2002, Virology; 303(1):15-32; and Poole et al., 2002, Virology; 303(1):33-46). Andrejeva et al. found that the V protein interacts with MDA5, resulting in a blockade of IFN-β expression (Andrejeva et al., 2004, Proc Natl Acad Sci USA; 101(49):17264-17269). In addition, they found that knocking down expression of MDA5 reduces IFN expression induced by poly(I):poly©, indicating that MDA5 plays an essential role in induction of IFN expression by dsRNA. In this example, the activation of IFN by rPIV5VΔC infection has been investigated and a viral mRNA with 5′-cap has been identified as an activator of IFN expression through a MDA5-dependent pathway that includes RNase L.

### Materials and Methods

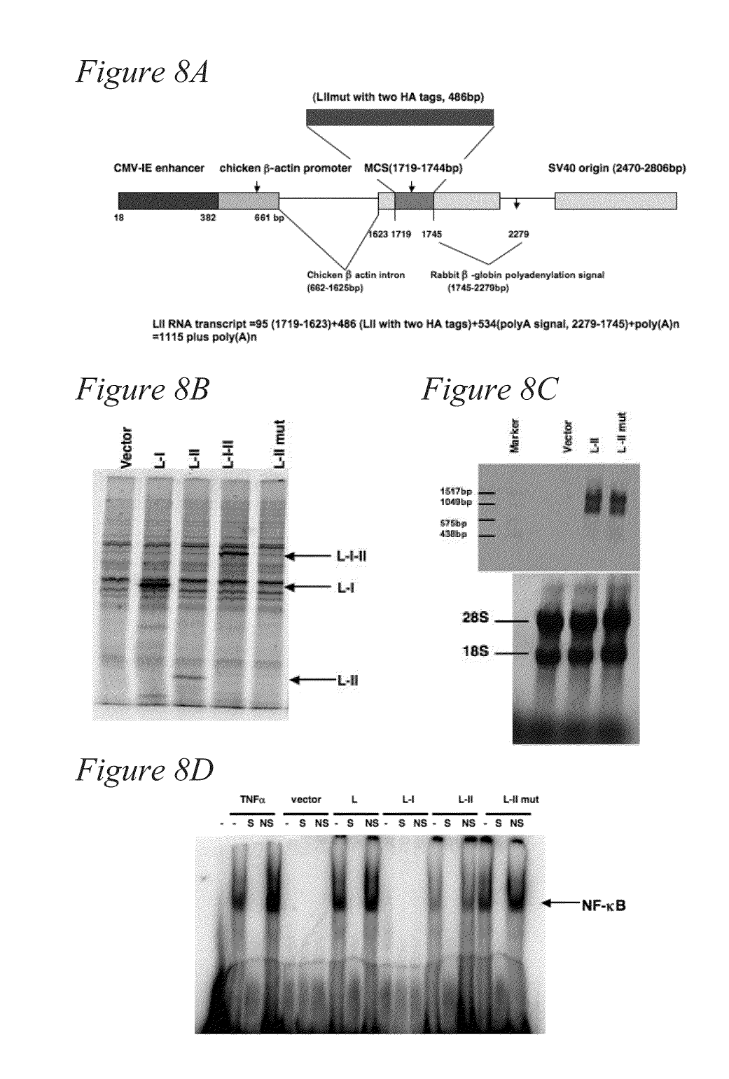

Cells and Plasmids. BSR-T7, HeLa, Vero, 293T, Huh 7.0, Huh 7.5 cells, and mouse embryonic fibroblasts (MEF) cells were cultured as previously described (Luthra et al., 2008, J Virol; 82(21):10887-95; Sumpter et al., 2005, J Virol; 79(5):2689-99; Malathi et al., 2007, Nature; 448(7155):816-9; and Buchholz et al., 1999, J Virol; 73(1):251-9). Plasmids encoding wild-type RNase L, RNase L mutant (R667A), L-I, L-I-II, PIV5 L, AKT1 with a Flag tag, the dominant negative (DN) mutant of AKT (pMT2-AH-AKT1, which contains 1-147 residues of AKT with a Myc antigen tag), the RIG-I DN (Flag-tagged RIG-I consisting of residues 218-925), the IPS-1 DN (with deletion of the CARD domain), phTK-RL, pNF-kB-TATA-F-Luc, and a plasmid containing F-Luc under control of an IFN-β promoter have been previously described (Poole et al., 2002, Virology; 303(1):33-46; Luthra et al., 2008, J Virol; 82(21):10887-95; Seth et al., 2005, Cell; 122(5):669-82; Yoneyama et al., 2004, Nat Immunol; 5(7):730-7; Malathi et al., 2007, Nature; 448(7155):816-9; Ling et al., 2009, J Virol; 83(8):3734-42; Sun et al., 2008, J Virol; 82(1):105-14; Sun et al., 2004, J Virol; 78(10):5068-78; and Lin et al., 2007, Virology; 368(2):262-72). Plasmids L-II (consisting of domain II) and L-II mut (consisting of STOP codon instead of START codon in a L-II background) with an antigenic tag (HA) in expression vector pCAGGS, were generated using standard molecular cloning techniques. A schematic of L-II mut is shown in FIG. 8A. Plasmids were prepared using a maxi prep kit from Qiagen. The endotoxin concentration was measured using a LAL endotoxin assay kit (GenScript). The endotoxin concentrations of all plasmids were lower than 0.1 EU/μg of DNA.

EMSA. BSR-T7 cells were transfected with vector or a plasmid encoding L, LI, or L-II, and nuclear extracts were prepared using a nuclear extraction kit (Marligen Biosciences). Nuclear extracts from TNF-α-treated BSR-T7 cells were used as a positive control. The cells were treated with 20 ng/ml of TNF-α for two hours (h). EMSA was carried out as previously described (Luthra et al., 2008, J Virol; 82(21):10887-95).

Dual Luciferase assay. Cells in 24-well tissue culture plates at 80-90% confluency were transfected. For BSR-T7 cells, the transfection was performed using Plus and Lipofectamine (Invitrogen), and for 293T or HeLa cells, transfections were performed using Lipofectamine 2000 (Invitrogen). Vector plasmid (pCAGGS) was used to maintain a constant total plasmid DNA per well. The amounts of plasmids were: 2.5 ng phRL-TK, 60 ng pNF-κB-TATA-F-Luc, and 240 ng pIFN-Luc. A range of concentrations up to 1,500 ng of plasmids encoding L, L-I, L-II, L-I-II, and L-II mut were used. The AKT DN, pMT2-AH-AKT, was used at 800 ng, and plasmid C-RIG (RIG-I DN) and IPS-1 DN were used at 500 ng. At 18-24 h after transfection, cells were lysed in 100 μl of passive lysis buffer (Promega) for 30-45 minutes. Twenty μl of lysate from each well was then used for dual luciferase assay using a Luminometer following manufacturer's protocol (Promega). To examine the effect of AKT inhibitor on L-activated NF-κB, 0.5 μM of AKT inhibitor (IV) was added to BSR-T7 cells, 4 hours after transfection.

Co-immunoprecipitation (Co-IP). To examine the interaction between L-II and AKT1, Co-IP was performed as previously described in Luthra et al. (Luthra et al., 2008, J Virol; 82(21):10887-95). Briefly, BSR-T7 cells transfected with a plasmid encoding AKT1 were immunoprecipitated with anti-AKT1 antibody, and then the precipitated AKT1 was used for further immunoprecipitation with L-I and L-II. L-I and L-II with HA tag were synthesized in vitro using TNT coupled transcription/translation systems (Promega) using ³⁵S[methionine/cysteine] labelling as previously described (Luthra et al., 2008, J Virol; 82(21):10887-95).

Immunoprecipitation and Immunoblotting. Cells transfected with plasmids encoding L-I, LII, LI-II, LII mut with a HA tag, or vector were metabolically labelled with [³⁵ S]Wet and [³⁵S]Cys for 3 h at 24 h post-transfection. The cell lysates were precipitated with anti-HA antibody. The precipitated proteins were resolved by 15% sodium dodecyl sulfate-polyacrylamide gel electrophoresis (SDS-PAGE) and visualized using a Storm Phosphorlmager (Molecular Dynamics). For immunoblotting, lysates from the luciferase assays were diluted (1:1) with protein lysis buffer (2% sodium dodecyl sulfate, 62.5 mM Tris-HCl pH 6.8, 2% dithiothreitol) and sonicated. Up to100 μl of the lysate was resolved in 10% SDS-PAGE and immunoblotting was performed using respective antibodies (Andrejeva et al., 2004, Proc Natl Acad Sci USA; 101(49):17264-17269).

RNA Purification and Transfection. 293T or Vero cells were transfected with empty vector, or a plasmid encoding L, L-I, L-II, LI-II, or L-II mut. Cells (HeLa or Vero) were infected with wild type PIV5 or rPIV5VΔC, or mock-infected. 18-20 h after transfection or infection, total RNA was isolated using a Qiagen RNeasy kit, or mRNA was isolated using Qiagen Oligotex direct mRNA purification kit, following manufacturer's instructions. Purified total RNA (1 μg/μl per well of 24-well plate) or mRNA (200 ng/μl per well of 24-well plate) was transfected into 293T cells using lipofectamine 2000. Poly(I):poly©) (500 ng/μl per well of 24-well plate) was used as a positive control. At one day post- transfection, IFN-β production was determined using a human IFN-β ELISA kit (PBL Interferon Source, NJ), following manufacturer's instructions.

Northern Blot Analysis. The RNA samples purified from BSR-T7 cells that were transfected with empty vector (pCAGGS), or a plasmid encoding L-I, L-II, or L-II mut for 18 h were electrophoresed on a 1.2% agarose gel in the presence of 0.44 M formaldehyde, transferred to a positively-charged nylon membrane (Roche Diagnostics), fixed by UV crosslinking, and analyzed by hybridization with DIG-labeled RNA probes that were generated by in vitro transcription using the DIG Northern Starter kit (Roche Applied Sciences). The hybridized probes were detected with anti-digoxigenin-AP Fab fragments and were visualized using chemiluminescence substrate CDP-Star on X-ray films The DNA templates for generating DIG-RNA probes were prepared using PCR with gene-specific sense oligomer and antisense oligomer with T7 RNA polymerase promoter sequence. The amplified PCR fragments were purified using a PCR purification column and gel purification kit (GenScript). A digoxigenin-labeled RNA molecular weight marker (Roche) was used to indicate the size of RNA.

RNase H treatment. The purified RNA from L-II-transfected cells or infected cells was used for a reverse transcription reaction using L-II specific primer or NP-specific primer. The RT products were treated with RNase H and purified using a RNeasy column. The treated or untreated RT product or L-II RNA were transfected into 293T cells and at one day post-transfection, the concentration of IFN-β in the medium was measured using ELISA.

In vitro RNA Transcription. DNA containing the region I or II of the L gene were amplified by PCR with sequence-specific sense primer containing a T7 polymerase promoter sequence and the antisense primer, using plasmid containing L-I or L-II as template. The L-I or L-II RNA fragments were in vitro synthesized using Riboprobe In vitro transcription systems (Promega). The synthesized fragments were treated with DNase Ito remove the DNA template and were then purified using a RNeasy column (Qiagen). The in vitro synthesized RNA fragments were treated with calf intestinal phosphate (CIP) for two hours and then purified. The purified CIP-treated in vitro RNA transcripts (200 ng) were transfected into 293T cells using Lipofectamine 2000. At one day post-transfection, IFN-β production was measured using a human IFN-β ELISA kit.

siRNA. Small interfering RNA (siRNA) experiments were performed as previously described (Luthra et al., 2008, J Virol; 82(21):10887-95; and Sun et al., 2008, J Virol;

82(1):105-14). Cells in 24-well plates at 30 to 50% confluency were transfected with 100 nM of siRNA purchased from Dharmacon [non-target siRNA pool (NT), MDA5 siRNA] and Santa Cruz (RNaseL, RIG-I siRNA) with the use of Oligofectamine (Invitrogen). At 48 h after siRNA transfection, the cells were transfected with empty vector, plasmids expressing L, LII, or LII mutant (1 μg/μl), or poly(I):poly(C)(500 ng/ml) using lipofectamine 2000, along with phRL-TK and pNF-κB-TATA-F-Luc or pIFN-Luc as previously described. At one day post-transfection, the dual luciferase assay and immunoblotting experiments were performed.

Enzyme Linked Immunosorbent Assay (ELISA) for IFN-β. Medium was collected and centrifuged to remove cell debris. 50 μl of the cleared medium or the IFN-β standard were used in duplicate for detection of IFN-β using a human IFN-β ELISA kit (PBL Interferon Source, NJ) following manufacturer's instructions.

Real-Time PCR. 293T cells in 6 well plates were transfected with 1 μg of purified RNA (vector, L-I, or L-II mut) or poly(I):poly(C) (250 ng) in OPTI-MEM using lipofectamine 2000 for 4 h. After transfection, the medium was changed to complete medium (10% FBS, 1% penicillin and streptomycin, DMEM) with DMSO or cyclohexamide (CHX; 20 μg/ml). After 16 hours incubation, the total RNA was isolated using RNeasy Mini kit (Qiagen). Eleven μl of total RNA for each sample was used for reverse transcription using Superscript III reverse transcriptase (Invitrogen) with oligo (dT)₁₅ according to manufacturer's protocol. The cDNA (4 μl of 1:20 diluted cDNA) from each sample was used for a real-time PCR reaction on a Step one Plus Real-Time PCR System (Applied Biosystems) using Taqman Universal PCR Master Mix (Applied Biosystems) and Taqman Gene Expression 1 Assays (Applied Biosystems) for IFN-β gene with FAM dye and β-actin gene with VIC dye. Results were analyzed to obtain Cₜ values. Relative levels of IFN mRNA and β-actin mRNA were determined by calculating ΔCₜ values. Each sample was run in three replicates.

### Results

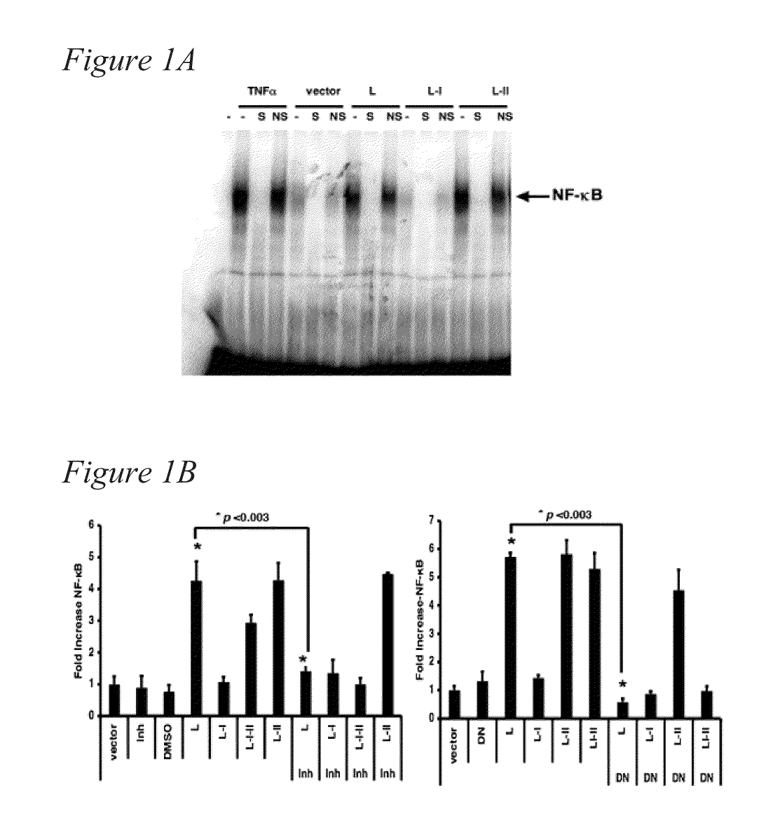

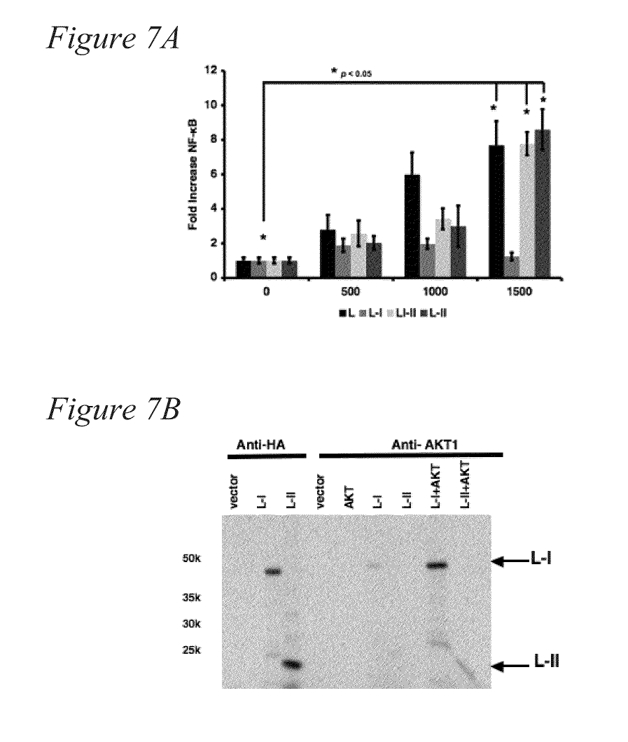

Region II of the L gene activated NF-κB independent of AKT1. Previously, it has been reported that the portion of the L gene containing the conserved regions I and II (L-I-II) together is sufficient to activate NF-κB (Luthra et al., 2008, J Virol; 82(21):10887-95). With this example, further deletion mutagenesis analysis of the L gene showed that region II, which contains 144 amino acid residues, was sufficient for the activation of NF-κB using an electrophoretic mobility shift assay (EMSA) (FIG. 1A). The result was confirmed using a reporter gene assay (FIG. 7A). This result was somewhat of a surprise since the previous report showed that activation of NF-κB by the L gene requires AKT1 and region I (L-I), which binds to AKT1 (Luthra et al., 2008, J Virol; 82(21):10887-95). The interaction between AKT1 and L-II was re-examined and confirmed that L-II did not bind to AKT1 (FIG. 7B). Interestingly, an AKT1 inhibitor, AKTIV, or an AKT1 dominant negative (DN) mutant, had no effect on activation of NF-κB by L-II (FIG. 1B), indicating that the L-II region activated NF-κB through a novel AKT1-independent mechanism.

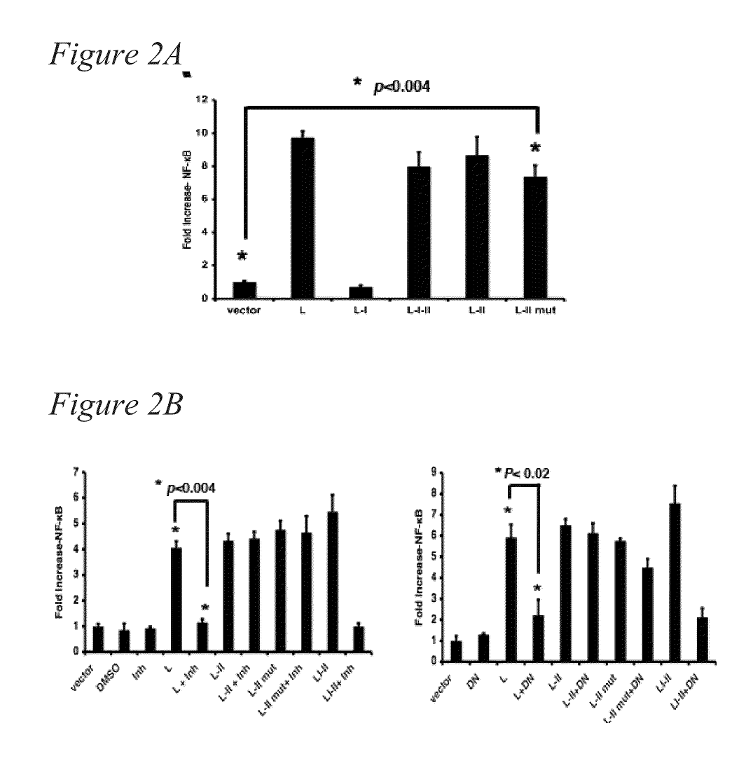

The RNA of region II of the L gene activated NF-κB. Because RNA can activate NF-κB (Randall and Goodbourn, 2008, Gen Virol; 89(Pt 1):1-47), it was suspected that the RNA sequence within the L-II region, not the amino acid residues encoded by the L-II region, might be responsible for NF-κB activation. The start codon of L-II was mutated into a stop codon (L-II mut) and found that the L-II mut did not express protein, although the expression levels of RNAs were similar between L-II and L-II mut (FIGS. 8A, 8B, and 8C). Interestingly, this mRNA generated from the plasmid pCAGGS, which is under the control of a pol-II promoter (Niwa et al., 1991, Gene; 108:193-200), activated NF-κB (FIG. 2A), and the result was confirmed by EMSA (FIG. 8D), suggesting that a mRNA of viral origin can activate NF-κB. Consistent with previous observations (FIG. 1B), this activation was not inhibited by the AKT1 inhibitor AKTIV or an AKT1 DN mutant (FIG. 2B).

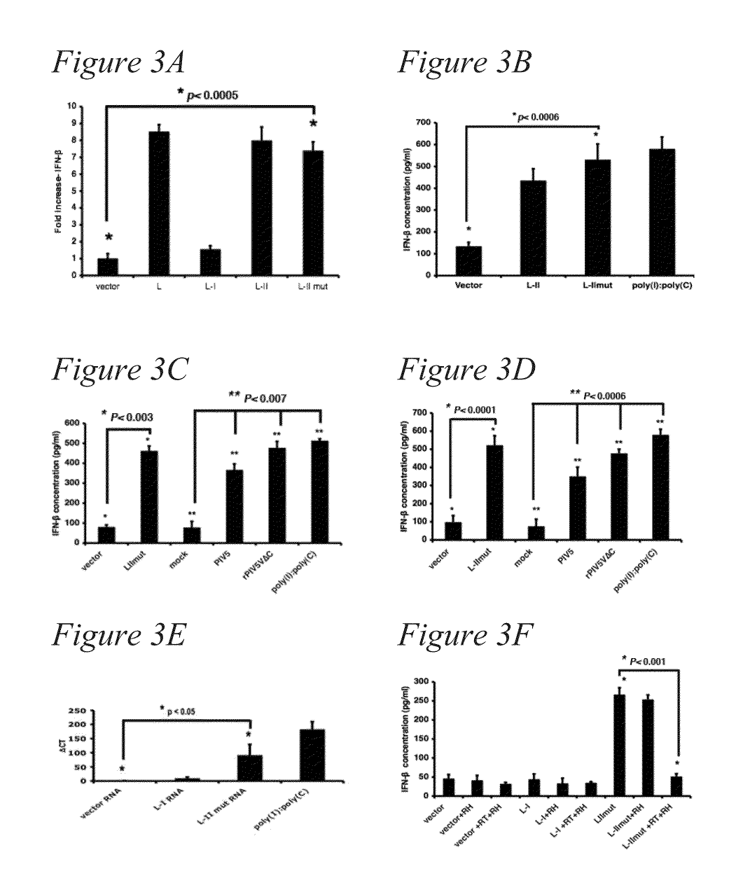

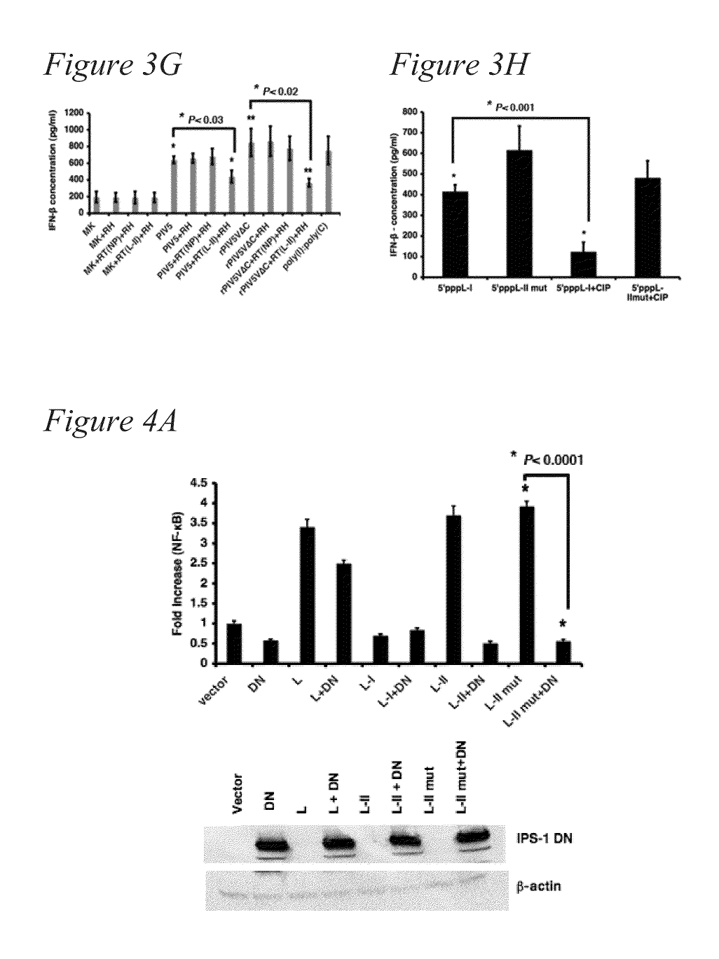

The RNA of region II of the L gene activated IFN-β expression. Because activation of NF-κB can lead to activation of IFN expression, the ability of this RNA to activate IFN expression was examined using a plasmid containing a reporter gene (F-Luc) under the control of an IFN-β promoter. As shown in FIG. 3A, the plasmid expressing the L-II mut RNA activated IFN-β promoter-driven reporter gene expression, suggesting that the RNA activated the IFN promoter. Furthermore, the amount of IFN-β in the medium of cells transfected with plasmids encoding L-II or L-II mut mRNAs were measured using ELISA. The plasmid encoding the L-II mut induced equivalent IFN-β production to the positive control, poly(I):poly(C) (FIG. 3B). To confirm that it is the RNA, not plasmid DNA, that activated IFN-β expression, the RNAs from transfected cells were purified and transfected into fresh cells, and levels of IFN-β in the medium of the RNA-transfected cells were measured after 1 day.

As shown in FIG. 3C, the RNA from the L-II mut-transfected cells produced a higher level of IFN-β than the RNAs from vector-transfected cells. Interestingly, both RNAs from wild-type and rPIV5VΔC-infected cells induced expression of IFN-β, indicating that RNAs capable of activating IFN-β expression exist in virus-infected cells as well (FIG. 3C). To further confirm that it was the mRNA that activated IFN-β expression, mRNAs from cells transfected with plasmids encoding L-II mut were purified and the mRNAs transfected into fresh cells. The amounts of IFN-β in the medium of cells transfected with mRNA from cells transfected with a plasmid expressing L-II mut mRNA were similar to that of those stimulated with poly(I):poly(C) (FIG. 3D), indicating that the mRNA activated expression of IFN-β.

The result that the L-II RNA activated IFN-β transcription in the presence of the protein synthesis inhibitor cyclohexamide (CHX) (FIG. 3E) indicates that the activation of IFN-β does not require new protein synthesis and that the L-II RNA activated IFN-β expression at the mRNA level. To validate the role of L-II mRNA in activating IFN-β expression, the mRNA was removed from the total RNA purified from the L-II plasmid- transfected cells by carrying out a reverse transcription (RT) reaction using a L-II specific primer, followed by treating the RT products with RNase H, which digests RNA in a RNA-DNA hybrid. This L-II mRNA-depleted mRNA did not stimulate production of IFN-β, indicating that L-II mRNA is essential for activation of IFN-β expression (FIG. 3F).

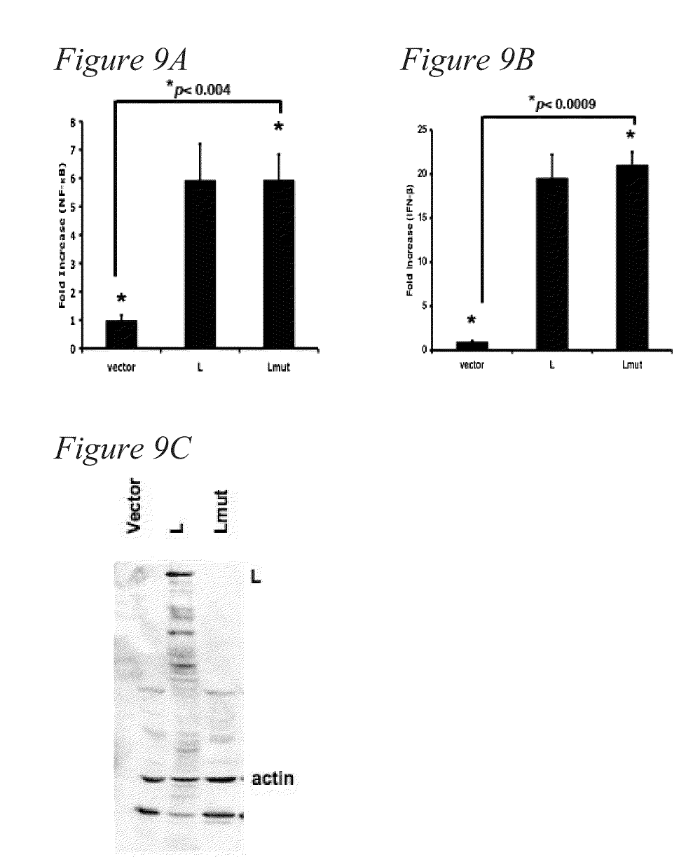

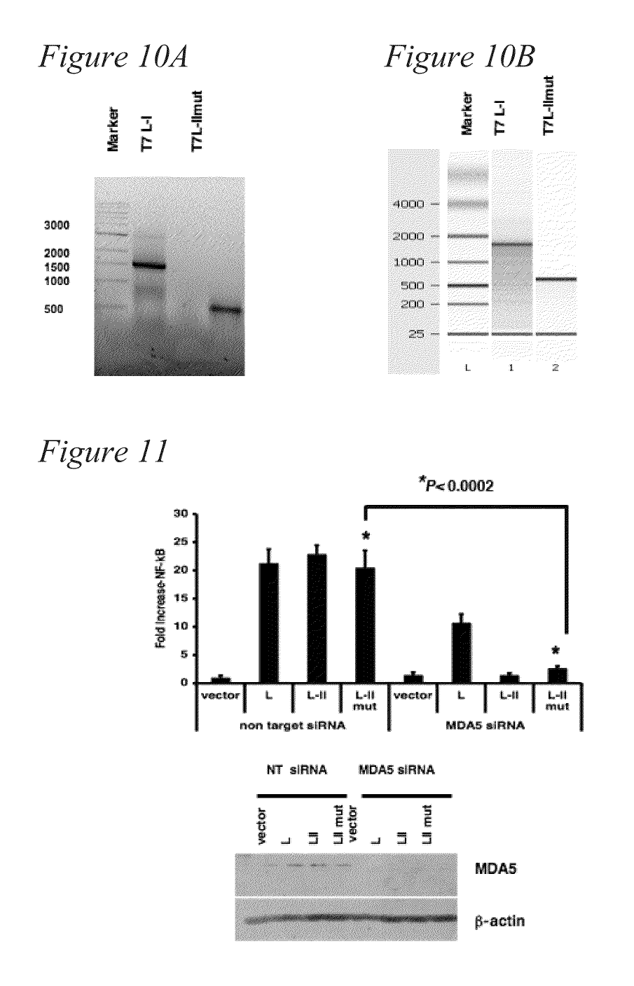

A similar experiment was carried out using RNA purified from virus-infected cells (FIG. 3G). Reverse transcription using a L-specific primer, but not NP-specific primer, reduced production of IFN-β, indicating that the L mRNA in virus infection is responsible for activating expression of IFN-β. Furthermore, a plasmid expressing a mutant L gene, with two stop codons placed downstream in-frame of its start codon, induced activation of NF-κB and IFN-β, confirming that the L mRNA is capable of activating expression of IFN-β (FIGS. 9A-9C). To determine whether the L-II RNA is capable of activating IFN-β expression by itself, L-II RNA was generated by in vitro transcription using T7 RNA polymerase (RNAP) (FIGS. 10A and 10B). RNAs from both the L-I and L-II region activated expression of IFN-β as expected (FIG. 3H), since T7 RNAP transcripts have 5′-triphosphate, a known activator of IFN through RIG-I. Interestingly, while removing 5′-triphosphate with calf intestinal phosphatase (CIP) reduced activation of IFN-β by the L-I RNA, this had minimal impact on the effect of L-II RNA, confirming that L-II RNA in its own right can activate IFN-β expression (FIG. 3H).

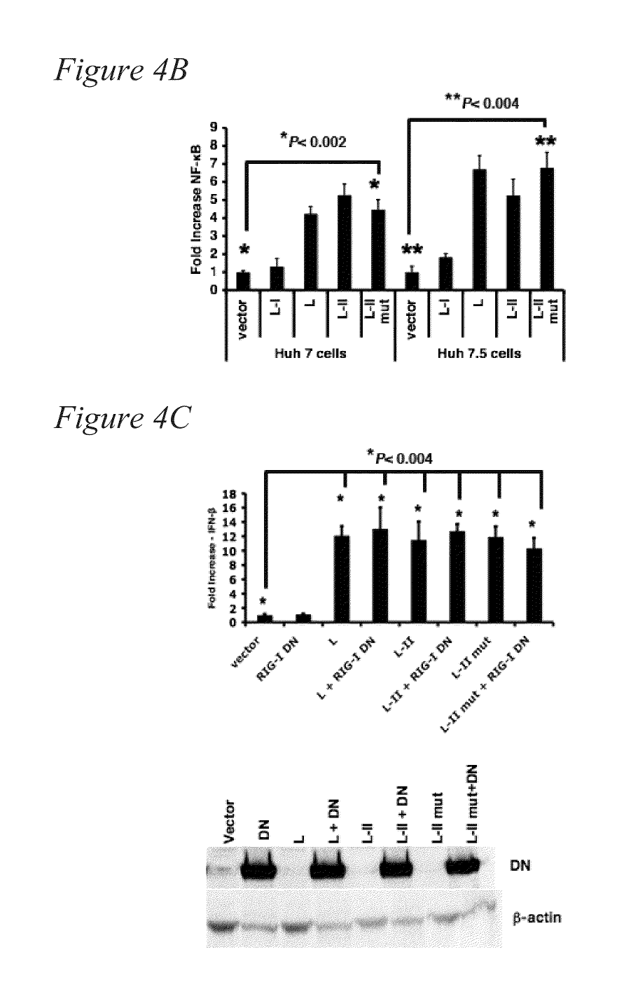

The RNA of region II of the L gene activated IFN-β expression through a MDA5-dependent pathway. There are two known cytoplasmic proteins that sense non-cellular RNA: RIG-I and MDA5 (Kato et al., 2006, Nature; 441(7089):101-5). Both of these proteins activate IFN expression through IPS-1 protein (Seth et al., 2005, Cell; 122(5):669-82; and Yoneyama et al., 2004, Nat Immunol; 5(7):730-7). Expression of a dominant negative (DN) mutant of IPS-1 blocked the activation of NF-κB by plasmids expressing L-II RNA (FIG. 4A), implying that RIG-I and/or MDA5 may play a role in L-II-induced, NF-κB activation. To determine whether these two proteins are involved in NF-κB activation by L-II RNA, plasmids encoding the L-II RNA were transfected into Huh7 or Huh7.5 cells, the latter having a defective RIG-I gene (Sumpter et al., 2005, J Virol; 79(5):2689-99). No difference in NF-κB activation was observed between the two cell lines, suggesting that RIG-I does not play a role in NF-κB signaling in response to L-II (FIG. 4B). Furthermore, RIG-I DN had no effect on the activation of NF-κB (FIG. 4C), confirming that RIG-I does not play a role in L gene-induced activation of NF-κB.

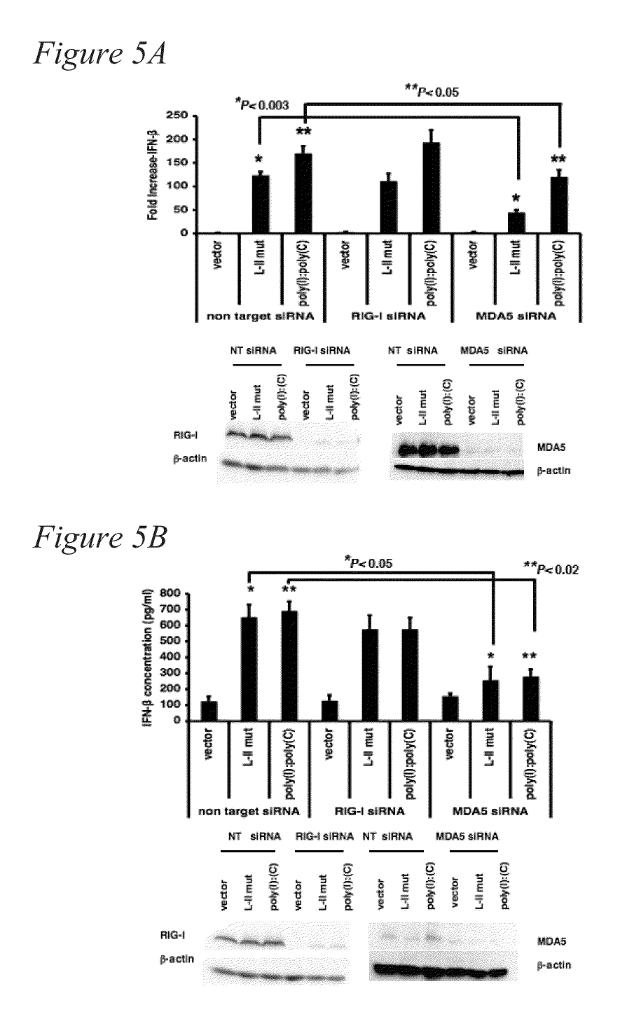

To investigate the role of MDA5, MDA5 expression was reduced using siRNA. This resulted in reduced NF-κB activation (FIG. 11), indicating that MDA5 plays a critical role in NF-κB activation. To further examine the roles of RIG-I and MDA5 in activating IFN-β expression, the effects of siRNA targeting RIG-I or MDA5 on IFN-β promoter activation were investigated. siRNA targeting MDA5, but not RIG-I, reduced IFN-β promoter activation by the plasmid expressing L-II mut RNA (FIG. 5A). This was further confirmed by examining IFN--β production by cells transfected with plasmids expressing L-II RNA after treatment with siRNA targeting RIG-I or MDA5. As shown in FIG. 5B, MDA5 siRNA reduced IFN-β production after plasmid transfection, whereas RIG-I siRNA had no significant effect, indicating that MDA5, but not RIG-I, plays a role in activating IFN-β expression by L-II RNA.

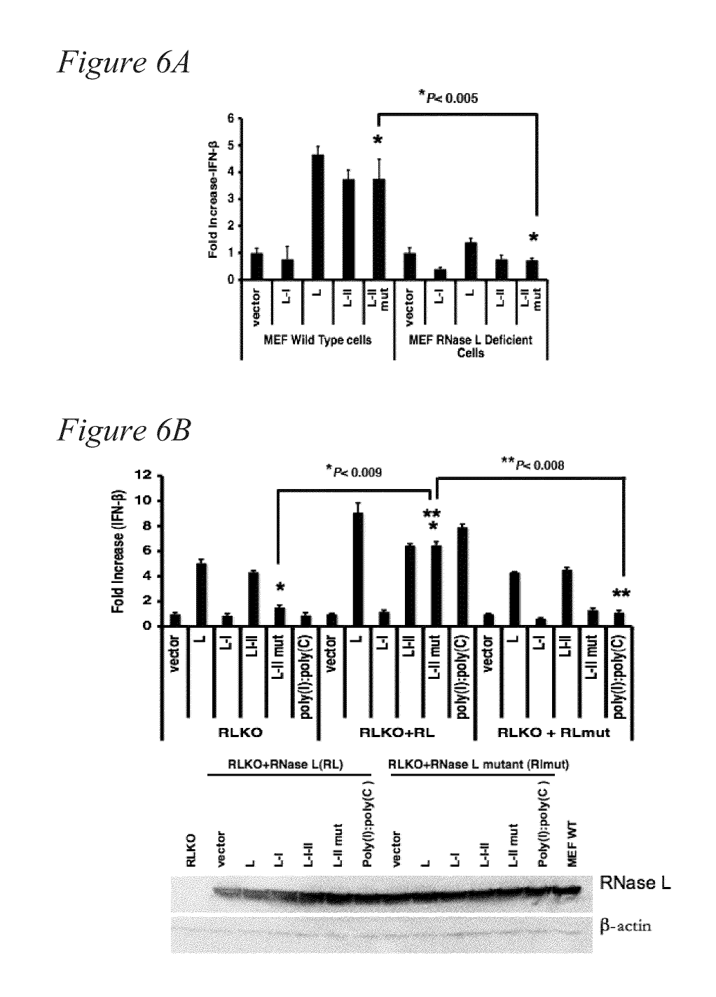

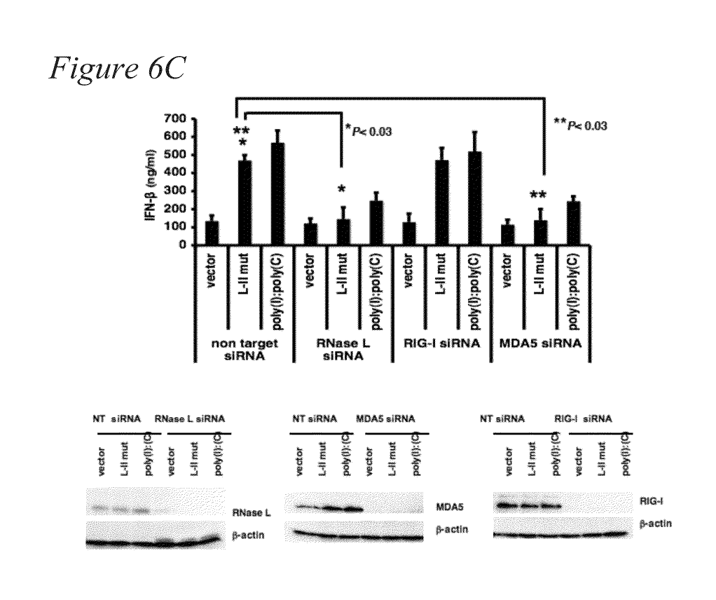

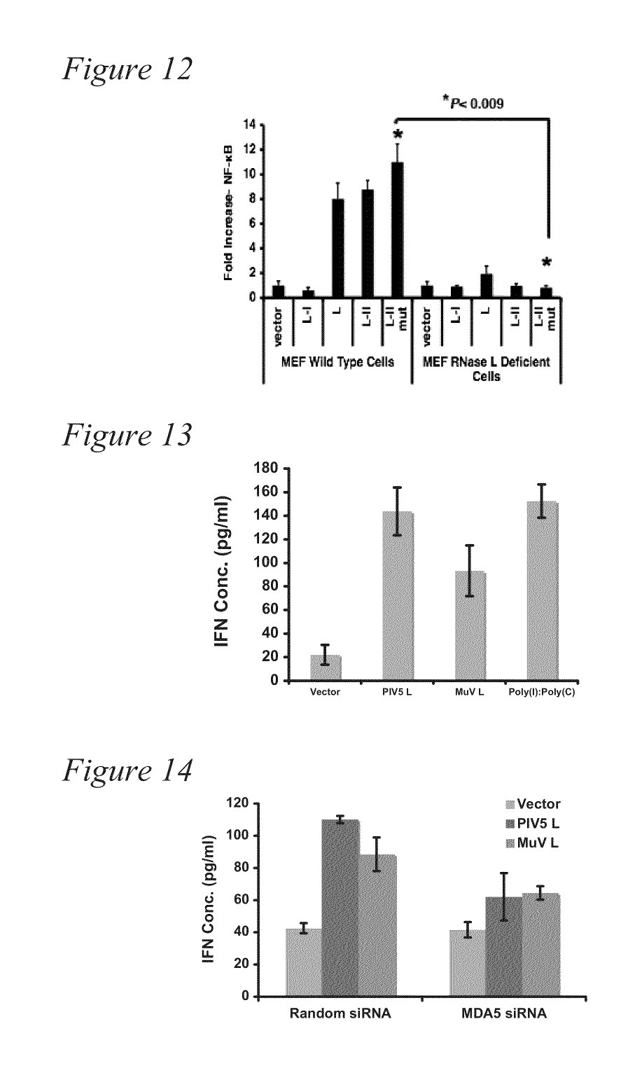

The RNA of region II of the L gene activated IFN-β expression through a RNase L-dependent pathway. Previously, it has been reported that RNase L plays an important role in IFN expression (Malathi et al., 2007, Nature; 448(7155):816-9). To examine the role of RNase L, mouse embryonic fibroblast (MEF) cells from mice expressing (WT) or deficient in RNase L (RLKO) were used. Plasmids expressing L-II RNA activated the NF-κB and IFN-β promoters in WT MEF, but not in RLKO MEF (FIG. 6A and FIG. 12), suggesting that RNase L plays an important role in activation of IFN-β by this viral mRNA. To confirm these results, WT RNase L or a defective RNase L lacking ribonuclease activity were transfected into RLKO MEF. The plasmid expressing the L-II RNA activated IFN-β in the presence of WT RNase L, but not in the presence of a defective RNase L, in MEF (FIG. 6B). To confirm the role of RNase L in activation of IFN-β expression by L-II RNA, effects of siRNA targeting RNaseL were examined. As shown in FIG. 6C, siRNA targeting RNaseL reduced production of IFN-β.

### Discussion

RIG-I and MDA5 are two well-known sensors of virus infection for induction of IFN expression. 5′-triphosphate is an activator for RIG-I. Viral genomic RNA of negative-stranded viruses, such as influenza virus and Sendai virus, which have a 5′-triphosphate, can activate IFN expression via a RIG-I-dependent pathway (Kato et al., 2006, Nature; 441(7089):101-5; Pichlmair et al., 2006, Science; 314(5801):997-1001; Loo et al., 2008, J Virol; 82(1):335-45; and Rehwinkel et al., 2010, Cell; 140(3):397-408). However, a natural activator for MDA5 has not been identified in the literature. Because MDA5 is required for induction of IFN by the positive stranded RNA virus encephalomyocarditis virus (EMCV), whose 5′ RNA is covalently linked to VPg, a polypeptide, thus avoiding detection by RIG-I, it is thought that MDA5 recognizes a viral RNA different from the 5-triphosphate RNA that is recognized by RIG-I (Kato et al., 2006, Nature; 441(7089):101-5). Since dsRNA such as poly(I):poly© can activate IFN expression through MDA5, it is thought that the activator for MDA5 is a double stranded RNA. Unlike the negative-stranded viruses influenza virus and Sendai virus, PIV5, a negative-stranded RNA virus, activated IFN-β expression through a viral mRNA-induced, RNase L/MDA5-dependent pathway, consistent with previous reports that PIV5 and some paramyxoviruses activate IFN expression via MDA5 (Gitlin et al., 2006, Proc Nall Acad Sci USA; 103(22):8459-64; and Yount et al., 2008, J Immunol; 180(7):4910-8). PIV5 replicates entirely in the cytoplasm (Lamb and Kolakofsky, Paramyxoviridae: The viruses and their replication, in Fields Virology (Fourth Edition), D. M. Knipe and P. M. Howley, Editors. 2001, Lippincott, Williams and Wilkins: Philadelphia) and viral mRNA would be readily accessed by RNase L/MDA5 proteins.

Because dsRNA is a known trigger of MDA5, it is speculated that dsRNA generated during viral genome replication might activate MDA5. Viral genomic RNA of paramyxovirus is tightly encapsidated by nucleocapsid protein and is resistant to RNase digestion, and inaccessible to other host proteins such as MDA5. Therefore it is not surprising that no double-stranded RNA (dsRNA) was detected in paramyxovirus infection using dsRNA-recognizing antibody (Weber et al., 2006, J Virol; 80(10):5059-64). In addition, since the nascent genome RNA is encapsidated, the double-stranded region of the viral RNA genome within the replication/transcription complex is small (greater than 2 kb is thought to be a MDA5 trigger). Pichlmair et al. reported that long dsRNA was not sufficient to activate IFN expression through a MDA5-dependent pathway (Pichlmair et al., 2009, J Virol; 83(20):10761-9), and higher-order RNA structures containing both dsRNA and ssRNA are activators of MDA5-dependent IFN expression. The nature of the higher-order RNA structure remains unidentified. The identification in this example of a viral mRNA that activates IFN-β expression through MDA5 is consistent with these results.

The results of this example indicate that RNase L plays a role in sensing viral RNA by MDA5, leading to activation of IFN-β. RNase L is an antiviral protein activated by 2′-5′ oligoadenylate (2-5A). 2-5A is produced by 2′,5′ oligoadenylate synthetase, the expression of which is induced by IFN (Silverman, 2007, J Virol, 81(23):12720-9). Activated RNase L cleaves viral mRNA and prevents viral replication (Silverman, 2007, J Virol, 81(23):12720-9). RNase L has recently been reported to amplify MDA5-dependent IFN expression through cleavage of cellular RNA (Malathi et al., 2007, Nature; 448(7155):816-9). This indicates that RNase L recognizes the viral mRNA and processes it into an activator of MDA5 leading to expression of IFN, since siRNAs targeting RNase L and MDA5 reduced activation of IFN-β expression by the viral mRNA. Alternatively, it is possible that viral mRNA activates MDA5-dependent IFN expression, and RNase L plays a role in amplifying IFN production.

It is known that wild-type PIV5 induces low levels of IFN expression and rPIV5VAC infection produces high levels of IFN (He et al., 2002, Virology; 303(1):15-32; and Poole et al., 2002, Virology; 303(1):33-46). Interestingly, RNAs purified from cells infected with PIV5 and rPIV5VΔC induced expression of high levels of IFN-β. This result is consistent with previous reports that the V protein of PIV5 can block induction of IFN induced by PIV5 infection (He et al., 2002, Virology; 303(1):15-32; and Poole et al., 2002, Virology; 303(1):33-46).

This example mapped the L RNA sequence to a 432 nt long region. Further analysis using RNA structure prediction programs indicates potential secondary structures within the sequence. Further detailed structure and function analysis of the sequence will define the sequence element and structure(s) within the viral mRNA that activate IFN-β expression through RNase L and MDA5. This sequence and structure(s) may serve as a prototype for other natural triggers of MDA5. This work has not only identified a novel trigger for MDA5, but also may lead to the discovery of small RNA molecules capable of activating IFN-β expression, which might be useful in anti-viral therapy.

The results of this example can now also be found in Luthra et al., “Activation of IFN-β; expression by a viral mRNA through RNase L and MDA5,” Proc Natl Acad Sci USA, 2011 Feb. 1; 108(5):2118-23(Epub 2011 Jan. 18).

### Example 2

### Activation of IFN-β by Other Viruses

The mumps virus (MuV) is a paramyxovirus closely related to PIV5. To examine whether the L gene of MuV can activate IFN-β expression, a plasmid containing the L gene of MuV was transfected into cells and purified RNA from the transfected cells. The RNA was then transfected into fresh cells and IFN-β expression was measured. Cells were transfected with plasmid vector, a plasmid expressing PIV5 L or MuV L mRNA. The RNAs were purified from the transfected cells and, then transfected into fresh cells. IFN-β concentrations were measured as described in Example 1. As shown in FIG. 13, mRNA from the cells transfected with MuV L activated expression of IFN-β, indicating that the L gene of MuV, like that of PIV5, activated IFN-β expression. The L gene of MuV will be further analyzed and the sequence and structural elements that are essential and/or sufficient for activation will be determined.

To investigate the role of MDA5 in the activation of IFN-β expression by the L gene of MuV, the effects of MDA5 knock down was examined next. Cells were transfected with plasmid vector, a plasmid expressing PIV5 L or MuV L mRNA. Meanwhile, another set of cells were transfected with siRNA. At two days after transfection, the RNAs from the plasmid-transfected cells were purified and, then transfected into the cells with siRNA. IFN-β concentrations were measured. As shown in FIG. 14, siRNA targeting MDA5 reduced the activation of IFN-β expression by the L gene of MuV, indicating that MDA5 played a critical role in the activation.

### Example 3

### Defining RNA Structure and Sequences Essential and/or Sufficient for Activating IFN Expression

With Example 1, the RNA sequence responsible for the activation of IFN-β was mapped to a 432 nucleotide (nt) long region. With this example a structural and functional analysis will be carried out to define the sequence and structural elements within the 432-nt region of viral mRNA responsible for activation of IFN-β expression. These sequences and structures will then serve as prototypes for the identification of other triggers of MDA5.

Deletion mutagenesis. A series of RNA deletions will be generated using in vitro T7 RNA transcription. First, a construct breaking the sequence in half (about 250 nts each) will be generated, and the ability of these RNAs in activating expression of IFN will be measured. If one half of the sequence is sufficient to activate IFN expression, further deletion analysis will be carried out to define the minimal sequence that is sufficient to activate IFN expression. If neither half activates IFN expression, smaller deletions (about 100 nts) from 5′ or 3′ end will be made and the abilities of the construct RNAs to activate IFN measured. Deletion mutagenesis will identify the minimal sequence required to activate IFN.

Bioinformatic approaches. It is possible that one or more structural elements within the RNA sequence play an important role in the activation of IFN. Using RNA structure prediction program RNAFOLD, a potential secondary structure, a stem-loop, within the sequence, has been identified (FIGS. 15A and 15B). Since RNase L plays a role in activation of IFN by the RNA, the 432-nt sequence was searched for potential targets for RNase L. Six potential RNase L target sites were identified. When the entire human genome sequence is searched, it was determined that for a 432-nt long sequence, on average, there should be only one potential RNase L target. These RNAs will be synthesized in vitro since the largest size is only about 70 nt long and the activation of IFN by these RNAs will be determined.

Incubating RNase L and RNA. Example 1 indicates that RNase L interacts with the RNA, resulting in the RNA being processed into a form that is capable of activating MDA5, leading to activation of IFN expression. Since RNase L is a RNase and can cleave RNA, purified RNase L will be incubated with the 432-nt long RNA and the incubated RNA resolved on a gel. If the 432-nt RNA is processed by the RNase L, i.e., smaller but distinguished bands appear in gel, the processed RNA will be purified and their sequences determined using 5′RACE.

Structure and function analysis of the RNA. The structure of target RNA sequence identified as essential and/or sufficient for activation of IFN will be probed using RNase mapping. If the sequence contains a potential stem-loop structure, treating the RNA with different RNases will reveal potential double-stranded RNA region, a potential stem region for a stem-loop structure. Furthermore, if a structure is identified, substitution mutagenesis will be carried out to determine the importance of the sequence. For instance, primary sequence will be changed without changing secondary structure by mutating GC pair in the putative stem region of a stem-loop structure to CG or AT pair.

Incubation of the 432-nt long RNA with RNase L will provide an empirical approach. The 432-nt long RNA will first be incubated with purified but not-activated RNase L. Incubation will also be performed in the presence of RNase L activator, 2-5A. If RNase L cleaves the RNA without 2-5A, it will indicate that the RNA can activate RNase L, and a novel activator for RNase L will be identified; and RNase activation of RNase L leads to processing its activator, resulting in a likely activator for MDA. It is likely that RNase L will cleave the 432-nt long RNA in the presence of 2-5A, its known activator, since activated RNase L is known to cleave RNA. This will allow for the determination of the sequences of the products and an examination of their abilities to activate IFN expression.

A variety of in vitro synthesized RNA will be used in these experiments. Since the RNA synthesized in vitro likely will have 5′-triphosphate, which activates IFN expression in a RIG-I dependent manner, Huh7.5 cells, whose RIG-I is defective, will be used initially, to avoid interference from the RIG-I pathway. Results will be confirmed in additional cell types. Results of the three complementary approaches discussed above will identify a sequence that is smaller than the 432-nt RNA that is sufficient to activate IFN expression.

### Example 4

### In Vivo Induction of IFN

This example will conduct in vitro studies in the murine model to determine the amount of interferon produced after stimulation with the 432-nt RNA IFN-activating agent, or a fragment or analog thereof. First, the 432-nt RNA, or a fragment or analog thereof, will be introduced into mice through three different routes, intranasal, intraperitoneal (IP) and intramuscular (IM). The amount of IFN in the sera of the animals at day 0 (pre-inoculation), day 1, day 2, day 3, day 5 and day 7 will be measured, as described in Example 1. For intranasal, IP, and IM inoculation, three escalating dosages of the plasmid DNA described in Example 1 (5 μg, 25 μg and 125 μg per mouse) will be used. For each dosage, six mice will be used. PBS (saline solution) will be used as baseline and a plasmid without the sequence encoding the RNA will be used as a vector control. The induction of IFN by plasmid DNA expressing the 432-nt RNA, or fragment or analog thereof, will be measured, as described in Example 1. Next, both IFN serum levels and the localized expression of IFN in the liver will be determined after delivery of plasmid DNA directly, in situ, into the liver. Three escalating dosages of the plasmid DNA described in Example 1 (5 μg, 25 μg and 125 μg per mouse) and controls (PBS and vector DNA) will be assayed. Injection will be directly into the liver of mice and the induction of IFN by plasmid DNA expressing RNA will be measured, as described in Example 1.

### Example 5

### In Vivo Induction After Intramuscular Injection

Balb/c mice in a group of 10 were injected intramuscularly with 200 μl of PBS, 200 μg of vector control plasmid DNA in 200 μl volume or 200 μg of plasmid encoding the mRNA (L-IImut) in 200 μl volume. Vector plasmid (expression vector pCAGGS) and the L-IImut plasmid (with a STOP codon instead of START codon in a background), are describe in more detail in Example 1.

At one, two, and three days post injection, blood samples were collected from the mice. Concentrations of IFN-beta in the sera from the samples were measured using ELISA, as described in Example 1. Differences between L-IImut and PBS or Vector are statistically significant (p<0.05). The concentrations of L-IImut-injected mice reached about 480 μg/ml (adjusting for dilution factor, 4).

With this example, the serum levels of IFN-beta detected after activation by the mRNA encoded by the L-IImut plasmid is unprecedented. This is in contrast to earlier attempts at expressing IFN-beta using gene delivery approaches. In all earlier attempts, although the effects of IFN beta could be observed, actual levels of IFN-beta were very low.

### Example 6

### NF-B Activation by Fragments of the LIImut transcript

FIG. 17 shows the polynucleotide sequence of the L-IImut transcript (SEQ ID NO:1) and the amino acid sequence (SEQ ID NO:2) encoded by this transcript. The sequence shown is a deoxyribonucleic sequence, but the sequence may also be a ribonucleotide sequence, in which thymine (T) bases are replaced by uracil (U) bases. The first TAA triple (UAA) is the stop codon of the L-IImut, introduced as described in Example 1. This stop codon may also be TAA, TGE, UAA, UGA, or UAG triplet. The polynuclotide sequence may further include one or more stop codons (TAG, TAA, TGE, UAA, UGA, and/or UAG) at the 3′ end.

Following methodologies described in Example 1, and as shown in FIG. 7A, the activation of NF-κB by various mutations of the region II of the L gene were tested. The various constructs tested are shown in FIG. 18. The level of activation of NF-κB by the various constructs is also shown in FIG. 18.

### Example 7

### Prevention of Respiratory Syncytial Virus (RSV) and Influenza A Virus Infection of Mice

All animal studies are reviewed and approved by the University of Georgia Investigational Animal Care and Use Committee. 6-8 weeks old female BALB/c mice (Harlan, Indianapolis, IN) in a group of more than five were injected intramuscularly with 100 μl of DNA plasmid containing the L-II region of the L gene (2 μg/μl), empty vector (2 μg/μl), or sterile PBS. One day after injection with plasmid or PBS, mice were infected intranasally with 100 μl of A/PR/8/34 (H1N1; 600 PFU). Naive mice inoculated with either virus were used as controls. Mice were anesthetized with Avertin (2, 2, 2 tribromoethanol) by intraperitoneal injection prior to infection. One day after infection sera are collected from all mice to measure IFN-β levels. Lungs were harvested from mice inoculated with AJPR/8/34 3 days post infection and viral titers assessed. All samples were frozen at −80° C. until all specimens could be assayed together to minimize biological variation.

To prepare lung tissue, 1 ml of PBS containing antibiotics and 0.5% bovine serum albumin was added to each sample. Samples are homogenized using the TissueLyser (Qiagen) then centrifuged at 10,000 rpm for 5 minutes. The TCID₅₀ was determined for lungs harvested from influenza infected mice as previously described. Briefly, 10-fold serial dilutions of samples from 10^{'11} to 10⁻⁶ were made in Modified eagles medium (MEM) with TPCK [L-(tosylamido-2-pheyl)ethyl chloromethyl ketone]-treated trypsin (Worthington Biochemical Corporation, Lakewood, N.J.) (1 μg/ml). Dilutions of each sample were added to Madin-Darby canine kidney (MDCK; ATCC) cells (4 wells/dilution; 200 μl/well) and the cells were incubated for 48 h at 37° C. The contents of each well were tested for hemagglutination and the TCID50 calculated by the Reed and Meunch method.

A 1.5 log reduction of virus was observed in the lungs of animal treated with the L-II RNA expressing plasmid over the vector alone, PB or no treatment groups. The intramuscular administration of a DNA plasmid containing the L-II region of the L gene was effective in inhibiting influenza virus replication in vivo.

The efficacies of intraperitoneal (IP), intravenous (IV) and intranasal (IN) inoculation of the plasmid DNA will also be tested.

Following this procedure the prevention of respiratory syncytial virus (RSV) infection of mice will also be tested, administering 50 μl of RSV A2 engineered to express luciferase (5×10⁵ PFU) and assessing viral titer five days post infection.

### Example 8

### Treatment of Respiratory Syncytial Virus (RSV) and Influenza A Virus Infection of Mice

6-8 weeks old female BALB/c mice (Harlan, Indianapolis, Ind.) in a group more than 5 are infected intranasally with 50 μl of either RSV A2 engineered to express luciferase (5×10⁵ PFU) or 100 μl of A/PR/8/34 (H1N1; 600 PFU). Naive mice inoculated with either virus are used as controls. Mice are anesthetized with Avertin (2, 2, 2 tribromoethanol) by intraperitoneal injection prior to infection. One day after infection, the mice are injected intramuscularly with 100 μl of DNA plasmid containing the L-II region of the L gene (2 μg/μl), empty vector (2 μg/μl), or sterile PBS. Lungs are harvested from mice inoculated with A/PR/8/34 3 days post infection and from mice inoculated with RSV 5 days post infection to assess virus titers. All samples are frozen at −80° C. until all specimens could be assayed together to minimize biological variation. It is expected that the L-II region will provide protection to the animals by delaying onset of illness and/or reduce virus infection. While IM injection will be tested first, the efficacies of IP, IV and IN inoculation of the plasmid DNA will also be tested.

### Example 9

### Treatment of Rabies Virus Infected Mice

15 BALB/c mice (6 to 8 weeks of age) are inoculated with wt rabies virus at 10 IMLD₅₀ intramuscularly (IM). At the same time, the mice (n=5) are injected intramuscularly with 100 μl of DNA plasmid containing the L-II region of the L gene (1 μg/μl), empty vector (1 μg/μl), or sterile PBS. Mice will be monitored daily for sign of illness. At days 3, 6 and 9 p.i., animals will be sacrificed and brains, spinal cords, as well as DRG collected for evaluation of viral antigen by immunohistochemistry and/or viral RNA by realtime-PCR. It is expected that the L-II region will provide protection to the animals by delaying onset of illness and/or reduce, even possibly eliminating virus infection.

### Example 10

### Testing Enhanced Immune Responses of Vaccine Candidate with IFN-β Inducer

15 BALB/c mice (6 to 8 weeks of age) are inoculated with a plasmid encoding the HA gene of H1N1 virus intramuscularly (IM). At the same time, the mice (n=5) are injected intramuscularly with 100 μl of DNA plasmid containing the L-II region of the L gene (1 μg/μl), empty vector (1 μg/μl), or sterile PBS. At 21 days post inoculation, blood samples will be collected from mice and titer of anti-H1 antibodies will be compared and H1-specific T cells will be measured. It is expected that the L-II region will enhance immunity of animals, i.e., more robust responses in antibody titers and T-cell responses in L-II injected animals.

### Example 11

### Reduction of Influenza Virus Titers in Lungs of Mice Pretreated with Plasmid Expressing L-IImut mRNA

To determine whether the mRNA expressed from a plasmid can reduce influenza virus replication in animals, a plasmid encoding the L-II RNA was injected intramuscularly in mice and the mice were infected the next day with L-IImut RNA (also referred to herein as “L-II RNA, as the stop codon in L-IImut is immaterial to the functionality of L-II). Procedures were as described in previous examples.

Results show that the mice injected with a plasmid expressing the L-II RNA had lower titers of influenza virus in the lungs at three days post infection (FIG. 18), indicating that the L-II RNA reduced influenza virus replication in vivo. Thus, this IFN-β inducing RNA has application for a therapy for virus infection.

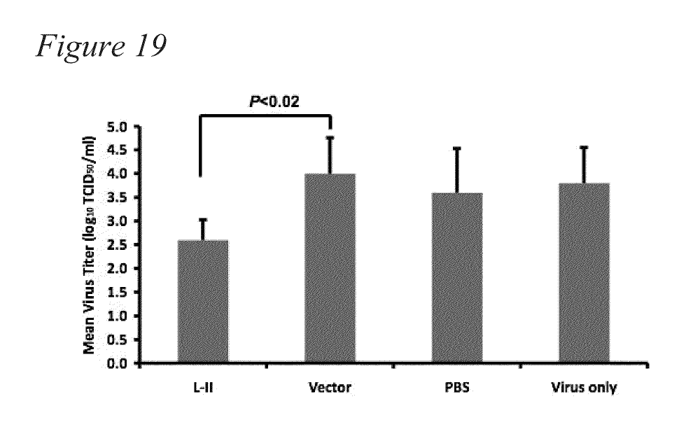

FIG. 19 shows the reduction of influenza virus by the L-II in vivo. 6-8 weeks old female BALB/c mice (Harlan, Indianapolis, Ind.) in a group more than 5 were injected intramuscularly with 100 μl of DNA plasmid containing the L-II region of the L gene (2 μg/μl), empty vector (2 μg/μl), or sterile PBS. One day after injection with plasmid or PBS, mice were infected intranasally 100 μl of A/PR/8/34 (H1N1; 600 PFU). Naive mice inoculated with either virus were used as controls. The TCID₅₀ was determined for lungs harvested from influenza infected mice and the TCID₅₀ was calculated by the Reed and Meunch method.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### Sequence Listing Free Text

SEQ ID NO:1 Polynucleotide sequence of L-IImut transcript

SEQ ID NO:2 Amino acid sequence encoded by L-IImut

What is claimed is:

1. A method of activating interferon beta expression in a subject and/or activating NF-κB expression in a subject, the method comprising delivering an isolated single stranded RNA sequence, the isolated single stranded RNA sequence comprising a nucleotide sequence that is at least about 90% identical to a nucleotide sequence encoding conserved region II of the L protein of a negative stranded RNA virus, or a fragment thereof, to the subject.

2. The method of claim 1 wherein the isolated single stranded RNA sequence comprises a nucleotide sequence that is at least about 90% identical to the nucleotide sequence SEQ ID NO:1, or a fragment thereof.

3. A method of treating a viral disease, cancer, and/or an autoimmune disease in a subject, the method comprising delivering an isolated single stranded RNA sequence to the subject, the isolated single stranded RNA sequence comprising a nucleotide sequence that is at least about 90% identical to a nucleotide sequence encoding conserved region II of the L protein of a negative stranded RNA virus, or a fragment thereof, wherein the isolated single stranded RNA sequence activates the expression of interferon beta and/or NF-κB in the subject.

4. The method of claim 3, wherein the isolated single stranded RNA sequence comprises a nucleotide sequence that is at least about 90% identical to the nucleotide sequence SEQ ID NO:1, or a fragment thereof, wherein the isolated single stranded RNA sequence activates the expression of interferon beta and/or NF-κB in the subject.

5. The method of claim 1, wherein the activation of IFN beta expression comprises IFN beta expression through a RNase L and/or MDA5-dependent pathway.

6. The method of claim 1, wherein the negative stranded RNA virus is of the family Paramyxoviridae.

7. The method of claim 6, wherein the virus of the family Paramyxoviridae is selected from the group consisting of human parainfluenza virus 1, human parainfluenza virus 2, human parainfluenza virus 3, human parainfluenza virus 4, parainfluenza virus 5, mumps virus, measles virus, human metapneumovirus, human respiratory syncytial virus, bovine respiratory syncytial virus rinderpest virus, canine distemper virus, phocine distemper virus, Newcastle disease virus, avian pneumovirus, Peste des Petits Ruminants virus (PPRV), Sendai virus, Menangle virus, Tupaia paramyxovirus, Tioman virus, Tuhokovirus 1, Tuhokovirus 2, Tuhokovirus 3, Hendravirus, Nipahvirus, Fer-de-Lance virus, Nariva virus, Salem virus, J virus, Mossman virus, and Beilong virus.

8. The method of claim 1, wherein the single stranded RNA sequence does not encode conserved region I of the L protein of a negative stranded RNA virus, and/or comprises a stop codon positioned so that the single stranded RNA is not translated into a polypeptide product.

9. The method of claim 1, wherein delivering the single stranded RNA sequence is by administering a DNA expression vector that transcribes the single stranded RNA sequence or by administering a composition comprising the single stranded RNA sequence.

10. The method of claim 9, wherein the single stranded RNA sequence is an mRNA with a 5′ cap.

11. The method of claim 1, wherein the fragment comprises at least 10 consecutive nucleotides of SEQ ID NO:1 and the fragment activates IFN beta expression through a RNase L and/or MDA5-dependent pathway.

12. The method of claim 11, wherein the polynucleotide sequence comprises a stop codon at its 5′ end and is not translated into an amino acid sequence.

13. The method of claim 1, wherein delivery is intramuscular, intranasal, intravenous, intreperitoneal, subcutaneous, and/or topical.

14. The method of claim 1, wherein delivery of the single stranded RNA sequence is regulated by a tissue specific promoter.

15. The method of claim 1, wherein delivery is to the mucosal membranes of the respiratory tract.

16. The method of claim 15, wherein delivery is by as aerosol.

17. The method of claim 1, wherein delivery is to liver, lung, central nervous system, nerves, muscle or tumor.

18. The method of claim 17, wherein delivery of the single stranded RNA sequence is regulated by a liver specific promoter.

19. The method of claim 18, wherein the subject has been exposed to Hepatitis C.

20. The method of claim 1, wherein the subject has been exposed to a viral disease, suffers from an autoimmune disease, or subject suffers from cancer.

21. The method of claim 20, wherein the cancer is melanoma.

22. A composition comprising:
as one aspect, one or more antigenic agents, and
as a second aspect, an isolated polynucleotide sequence comprising a nucleotide sequence that transcribes a single stranded RNA sequence comprises a nucleotide sequence that is at least about 90% identical to a nucleotide sequence encoding conserved region II of the L protein of a virus of a negative stranded RNA virus, or a fragment thereof.

23. The composition of claim 22, wherein the isolated polynucleotide sequence comprises a nucleotide sequence that transcribes a single stranded RNA sequence comprises a nucleotide sequence that is at least about 90% identical to the nucleotide sequence SEQ ID NO:1, or a fragment thereof, wherein the fragment thereof comprises at least 10 consecutive nucleotides of SEQ ID NO:1 and the fragment activates IFN beta expression through a RNase L and/or MDA5-dependent pathway a nucleotide sequence encoding conserved region II of the L protein of a virus of a negative stranded RNA virus, or a fragment thereof.

24. The composition of claim 22, wherein the negative stranded RNA virus is of the family Paramyxoviridae.

25. The composition of claim 22, wherein the single stranded RNA sequence does not comprise sequences encoding the conserved region I of the L protein of a negative stranded RNA virus, and/or comprises a stop codon positioned so that the single stranded RNA is not translated into a polypeptide product.

26. The composition of claim 22, wherein the antigenic aspect comprises a nucleotide sequence encoding an antigen.

27. An isolated polynucleotide sequence comprising a nucleotide sequence that is at least about 90% identical to a nucleotide sequence encoding conserved region II of the L protein of a negative stranded RNA virus, or a fragment thereof, wherein the isolated polynucleotide sequence activates IFNS beta expression through a RNase L and/or MDA5-dependent pathway, and wherein the isolated polynucleotide sequence comprises a stop codon positioned so that the single stranded RNA is not translated into an amino acid sequence and/or does not comprises sequences encoding the conserved region I of the L protein of a negative stranded RNA virus.


SEQUENCE LISTING (2 sequences)

SEQ ID NO: 1 - DNA, 426 bp, artificial
  polynucleotide encoding encoding an L-II mutant
taaatcgatagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggg

SEQ ID NO: 2 - protein, 141 aa, artificial
  L-II mutant polypeptide
IDRRSLIKQRHQHHQVPLPNPFNRRLLLNFLGDDKFDPNVELQYVTSGEYLHDDTFCASYSLKEKEIKPDGRIFAKLTKRMRSCQVIAESLLANHAGKLMKENGVVMNQLSLTKSLLTMSQIGIISEKARKSTRDNINQPG